(19) United States
(12) Patent Application Publication
(10) Pub. No.: US 2019/0055267 A1
(43) Pub. Date: Feb. 21, 2019

(54) ORGANOTRIFLUOROBORATE MIMICS OF AMINO ACIDS AND USES THEREOF

(71) Applicant: The United States of America, as represented by the Secretary, Department of Health and Human, Bethesda, MD (US)

(72) Inventors: Zhibo Liu, Chevy Chase, MD (US); Xiaoyuan Chen, Potomac, MD (US)

(73) Assignees: The United States of America, as represented by the Secretary, Department of Health and Human, Bethesda, MD (US); and Human Services, Bethesda, MD (US)

(21) Appl. No.: 15/570,546

(22) PCT Filed: Apr. 29, 2016
(86) PCT No.: PCT/US2016/030106

§ 371 Date: Oct. 30, 2017

Related U.S. Application Data
(60) Provisional application No. 62/155,085, filed on Apr. 30, 2015.

(51) Int. Cl. C07F 5/02; A61K 51/04; A61B 6/03; A61B 6/00

(52) U.S. Cl. CPC C07F 5/02; C07F 5/025; C07B 2200/05; A61B 6/037; A61B 6/50; A61K 51/04

(57) ABSTRACT

Disclosed are boron mimics of amino acids which are compounds, salts, solvates, or stereoisomers of the Formula (I): wherein A, R¹, R², and R³ are defined herein, and processes for the preparation of the compounds, salts, solvates, or stereoisomers. The compounds, salts, solvates, or stereoisomers of Formula (I) may be used in boron neutron capture therapy. The compounds, salts, solvates, or stereoisomers of Formula (I) with at least one fluorine being, ¹⁸F are useful for imaging tumors using positron emission tomography and for evaluating the treatment potential of anti-cancer drugs.

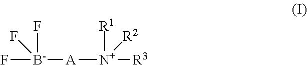

### CROSS-REFERENCE TO RELATED APPLICATIONS

This application is a U.S. National Phase of International Patent Application No. PCT/US2016/030106, filed Apr. 29, 2016, which claims the benefit of U.S. Provisional Patent Application No. 62/155,085, filed Apr. 30, 2015, each of which is incorporated herein by reference in its entirety.

### STATEMENT REGARDING FEDERALLY SPONSORED RESEARCH AND DEVELOPMENT

This invention was made with Government support under project number Z01EB000073 by the National Institutes of Health, National Institute of Biomedical Imaging and Bioengineering. The Government has certain rights in the invention.

### BACKGROUND OF THE INVENTION

Alpha-amino acids are the building-blocks of proteins and play roles in ATP production and neurotransmission. Alpha-amino acids also are key nutrients that cancer cells use for survival and proliferation. There is a direct relationship between uptake of amino acids and cancer cell replication, where the uptake is extensively upregulated in most cancer cells. This uptake increases as cancer progresses, leading to greater uptake in high-grade tumors and metastases. Amino acids act as signaling molecules for enhancing proliferation and also play a role in cancer-associated reprogrammed metabolic networks in the buildup of biomass.

Amino acids are desirable for use as PET tracers in cancer diagnosis. Amino acids are the basic building block for protein synthesis, and proliferation of tumors would not be possible without added uptake of amino acids. Unlike healthy tissue, many types of cancer cells need to use amino acids as an energy supply. Also, certain amino acids (e.g., Ile and Val, and especially Leu) stimulate mTOR (mechanistic target of Rapamycin) and assist acceleration of cancer cell growth.

Enormous effort has been devoted to developing radiolabeled amino acid probes, e.g., for use in positron emission tomography (PET), but the achievements have been rather limited. For example, a common limitation to utilizing most of the naturally occurring amino acids is susceptibility to in vivo metabolism, which may decrease tumor specificity and complicate kinetic analysis.

The primary radionuclides for amino acid tracers are ¹¹C (t_{1/2}=20.3 min) and ¹⁸F (t_{1/2}=109.8 min); however, the short half-life of ¹¹C necessitates an onsite cyclotron and requires the use of the radiotracer soon after preparation, making the routine clinical use of ¹¹C-labeled amino acids logistically difficult. In addition, as one of essential amino acids, ¹¹C-Leu is continually taken by healthy tissues for protein synthesis, and high-level uptake of ¹¹C-Leu is often observed in normal tissues whereas uptake in tumors is not always sufficiently high.

Shortcomings associated with ¹¹C-labeling were expected to be overcome with non-natural ¹⁸F-labeling; however, ¹⁸F also presented problems. No general method was available to label amino acids with ¹⁸F due to the challenging chemistry, where ¹⁸F could only potentially be introduced into the side-chain of the amino acid. This resulted in a non-natural amino acid often having reduced targeting specificity. Also, the problem of in vivo metabolism cannot be solved by simply utilizing these related non-natural amino acids, since the ¹⁸F-modified amino acids can still participate in regular protein synthesis through the formation of amide bonds (—CONH—).

There is an unmet need for traceable amino acid mimics, which may be non-invasively detected by imaging technology, including for clinical diagnosis and anti-cancer drug evaluation. There is also an unmet need for additional compounds and methods for the treatment of cancer.

### BRIEF SUMMARY OF THE INVENTION

The present invention provides, in one embodiment, a compound of the Formula (I):

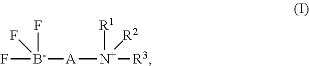

wherein A, R¹, R², and R³ are defined herein, and pharmaceutically acceptable salts, solvates, or stereoisomers thereof.

In another embodiment, the present invention provides a process for the preparation of the compounds, salts, solvates, or stereoisomers described herein.

In another embodiment, the present invention provides a method of imaging a tumor within a subject comprising administering to the subject an effective amount of a compound, salt, solvate, or stereoisomer with at least one fluorine being ¹⁸F, or a composition thereof, and utilizing positron emission tomography to take an image of the tumor within the subject. In another embodiment, the method further comprises taking more than one image of the tumor, wherein the images are taken at different times, and measuring the size of the tumor on each image. Using such a method, the effectiveness of potential anti-cancer drugs may be evaluated.

In another embodiment, the present invention provides a method of imaging tumor uptake, the method comprising identifying an amino acid having at least one COO⁻ moiety, generating a boramino acid mimic, wherein the boramino acid mimic has the same structure as the amino acid except a COO⁻ moiety of the amino acid is replaced with a BF₃⁻ moiety in the boramino acid mimic, wherein at least one fluorine is ¹⁸F, administering to a subject with a tumor an effective amount of the boramino acid mimic, and utilizing positron emission tomography to take an image of the tumor within the subject.

In another embodiment, the present invention also provides a method of treating a tumor in a subject, the method comprising administering to the subject an effective amount of a compound, salt, solvate, stereoisomer, or composition thereof as described herein and irradiating the subject with epithermal neutrons.

### BRIEF DESCRIPTION OF THE DRAWINGS

FIG. 1 presents HPLC radioactive traces of Sep-Pak-purified ¹⁸F-boramino acids in accordance with embodiments of the invention. The compounds are shown left to right in the order of elution.

FIG. 2 is a schematic depiction of system A, system L, and system P transporters. Leucine and phenylalanine are mainly transported by the system L transporters (also known as L-type or large amino acid transporters (LATs, e.g., LAT-1, -2, -3, or -4) or Leucine amino acid transporters), which exchange one amino acid (AA) from the extracellular compartment with one AA from the intracellular compartment and does not require Nat Alanine is mainly transported by the system A transporter which co-transports one extracellular amino acid with one Na⁺ into the cell. Proline is specifically transported by the system P transporters (one has a low K_{M} value (˜100 μM) and the other has a high K_{M} value (˜5000 μM)), which also co-transport one extracellular AA with one Na⁺ into the cell.

FIG. 3 is a line graph showing cell uptake (% AD) in U87MG cells of various boramino acids (BAA) over time in accordance with embodiments of the invention.

FIG. 4 is a bar graph showing intracellular ¹⁸F-Phe-BF₃ content of U87MG cells when in the presence of natural amino acids or amino acid transporter inhibitors in accordance with embodiments of the invention. Statistical significance was determined by one way ANOVA+Tukey's post hoc test, **p<0.01.

FIG. 5 is a bar graph showing intracellular ¹⁸F-Leu-BF₃ content of U87MG cells when in the presence of natural amino acids or amino acid transporter inhibitors in accordance with embodiments of the invention. Statistical significance was determined by one way ANOVA+Tukey's post hoc test, ***p<0.001.

FIG. 6 is a bar graph showing intracellular ¹⁸F-Ala-BF₃ content of U87MG cells when in the presence of natural amino acids or amino acid transporter inhibitors in accordance with embodiments of the invention. Statistical significance was determined by one way ANOVA+Tukey's post hoc test, **p<0.01.

FIG. 7 is a bar graph showing intracellular ¹⁸F-Pro-BF₃ content of U87MG cells when in the presence of natural amino acids or amino acid transporter inhibitors in accordance with embodiments of the invention. Statistical significance was determined by one way ANOVA+Tukey's post hoc test, **p<0.01.

FIG. 8 is a line graph showing time-dependence of ¹⁸F-Leu-BF₃ uptake in cells in accordance with embodiments of the invention.

FIG. 9 is a bar graph showing intracellular ¹⁸F-Leu-BF₃ content of UM22B cells when in the presence of natural amino acids or amino acid transporter inhibitors in accordance with embodiments of the invention. Statistical significance was determined by one way ANOVA+Tukey's post hoc test, ***p<0.001.

FIG. 10 is a line graph showing a nearly linear relationship is found between the cellular uptake of ¹⁸F-Leu-BF₃ in HEK293 cells and LAT-1 expression in accordance with embodiments of the invention.

FIG. 11 shows an illustration of transporter-mediated cell uptake of boramino acids in accordance with embodiments of the invention.

FIG. 12 shows an uptake-concentration curve of ¹⁸F-Phe-BF₃ in U87MG cells in accordance with embodiments of the invention.

FIG. 13 shows an uptake-concentration curve of ¹⁸F-Leu-BF₃ in U87MG cells in accordance with embodiments of the invention.

FIG. 14 shows an uptake-concentration curve of ¹⁸F-Ala-BF₃ in U87MG cells in accordance with embodiments of the invention.

FIG. 15 shows an uptake-concentration curve of ¹⁸F-Pro-BF₃ in U87MG cells in accordance with embodiments of the invention.

FIG. 16 is a line graph showing metabolic stability of ¹⁸F-Leu-BF₃ in accordance with embodiments of the invention.

FIG. 17 diagrammatically shows a maximum-intensity projection image of ¹⁸F-Phe-BF₃ in UM22B-bearing mice in accordance with embodiments of the invention.

FIG. 18 diagrammatically shows a maximum-intensity projection image of ¹⁸F-Leu-BF₃ in UM22B-bearing mice in accordance with embodiments of the invention.

FIG. 19 shows the biodistribution of ¹⁸F-Phe-BF₃ in selected organs at 120 min after injection (n=4) in accordance with embodiments of the invention. Statistical significance was determined by one way ANOVA+Tukey's post hoc test, *p<0.05, **p<0.01.

FIG. 20 shows the biodistribution of ¹⁸F-Leu-BF₃ in selected organs at 120 min after injection (n=4) in accordance with embodiments of the invention. Statistical significance was determined by one way ANOVA+Tukey's post hoc test, *p<0.05, **p<0.01.

FIG. 21 shows time-activity curves of ¹⁸F-Phe-BF₃ uptake in tumor and other tissues from a tumor-bearing mouse in accordance with embodiments of the invention.

FIG. 22 shows time-activity curves of ¹⁸F-Leu-BF₃ uptake in tumor and other tissues from a tumor-bearing mouse in accordance with embodiments of the invention.

FIGS. 23A and B diagrammatically show whole-body maximum intensity projection PET imaging of a UM22B-bearing mouse showing the uptake of radioactive ¹¹C-Leu (A) and ¹⁸F-Leu-BF₃ (B) in accordance with embodiments of the invention. The right image was artificially mirrored for better illustration. Scale bar is calibrated in % ID/g, with no background subtracted.

FIG. 24 shows time-activity curves of ¹¹C-Leu uptake in tumor and other tissues from a tumor-bearing mouse in accordance with embodiments of the invention.

FIG. 25 shows time-activity curves of ¹⁸F-Leu-BF₃ uptake in tumor and other tissues from a tumor-bearing mouse in accordance with embodiments of the invention.

FIGS. 26A-D diagrammatically show whole-body maximum intensity projection PET imaging of a UM22B-bearing mouse showing the uptake of radioactive ¹¹C-Leu (A), ¹⁸F-Leu-BF₃ (B), and ¹⁸F-FDG (C) in accordance with embodiments of the invention. Panel (D) shows that ¹⁸F-Leu-BF₃ demonstrates low uptake in healthy brain tissues comparing with natural Leu and ¹⁸F-FDG, in accordance with embodiments of the invention. Scale bar is calibrated in % ID/g, with no background subtracted.

FIG. 27 shows time-activity curves of ¹⁸F-Leu-BF₃, ¹¹C-Leu, and ¹⁸F-FDG uptake in inflammation and muscle of mice in accordance with embodiments of the invention.

FIG. 28 shows the biodistribution of ¹⁸F-Leu-BF₃, ¹¹C-Leu, and ¹⁸F-FDG in selected organs at 60 min after injection in accordance with embodiments of the invention.

FIG. 29 diagrammatically shows PET imaging of mice after varying injections of ¹⁸F-Leu-BF₃. The images were taken at 60 min post injection, and they are representative image from different mice.

FIG. 30 shows consistent tumor uptake of ¹⁸F-Leu-BF₃ at the level of 10% ID/g 60 min after the injection of <1 μg, 200 μg, 1 mg, 5 mg and 25 mg of ¹⁸F-Leu-BF₃, as determined by PET imaging, in accordance with embodiments of the invention.

FIG. 31 shows boron concentration may reach up to 30 ppm in tumor (based on calculation) at 60 min post-injection in accordance with embodiments of the invention.

FIG. 32 shows that after injection of ¹⁸F-Leu-BF₃, there is high boron accumulation in tumor and low uptake in brain and muscle at 60 min based on inductive coupled plasma (ICP) analysis in accordance with embodiments of the invention.

### DETAILED DESCRIPTION OF THE INVENTION

It has been surprisingly and unexpectedly found that replacement of the carboxylate moiety (—COO⁻) of an amino acid with a trifluoroborate moiety (—BF₃⁻), which is an isostere of the carboxylate group, gives a new type of compound: boramino acids (or BAA) that overcome problems as discussed above. Boramino acids may be designed, e.g., as mimics of naturally occurring α-amino acids, to be generally applicable for studies involving natural amino acids; may be designed to be metabolically stable (stable in vivo and also orthogonal to regular metabolism such as, for example, protein synthesis and ATP production); and may be designed to be non-distinguishable to natural amino acid transporters compared to natural amino acids. BAAs allow for the use of ¹⁸F as the radiolabel, which is a preferred isotope because, for example, of its ubiquity in general hospitals and utility for PET imaging. BAAs also allow for easy one step radiolabeling, where the step is in aqueous solution and does not require separation, such as through the use of HPLC. Thus, the radiolabeling of BAAs may be achieved through the use of a simple kit.

In one embodiment, the present invention provides a compound of the Formula (I):

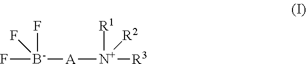

wherein A is optionally substituted alkylenyl (an alkylenyl group that is optionally substituted), wherein the substituents are selected from halo, alkoxyl, cycloalkyl, heterocycloalkyl, aryl, heteroaryl, hydroxyl, COOR², NR²R³, CONR²R³, SR², guanidino, alkynyl, and azido, wherein each substituent cycloalkyl, heterocycloalkyl, aryl, and heteroaryl is optionally fused with one or more groups selected from cycloalkyl, heterocycloalkyl, aryl, and heteroaryl, wherein each substituent alkoxyl, cycloalkyl, heterocycloalkyl, aryl, and heteroaryl is optionally further substituted with one or more substituents selected from halo, alkyl, cycloalkyl, heterocycloalkyl, aryl, heteroaryl, hydroxyl, COOR², NR²R³, CONR²R³, SR², guanidino, alkynyl, and azido, wherein substituent guanidino is optionally further substituted with one or more substituents selected from alkyl, cycloalkyl, heterocycloalkyl, aryl, and heteroaryl, wherein each of R¹, R², and R³ is independently H or alkyl, the alkyl optionally substituted with one or more substituents selected from halo, alkoxyl, cycloalkyl, heterocycloalkyl, aryl, heteroaryl, hydroxyl, COOR², NR²R³, CONR²R³, SR², guanidino, alkynyl, and azido; or A and R¹ together with the nitrogen of the NR¹R²R³⁺ moiety form a heterocycloalkyl or heteroaryl ring, wherein the ring is optionally substituted with one or more substituents selected from halo, alkyl, alkoxyl, cycloalkyl, heterocycloalkyl, aryl, heteroaryl, hydroxyl, COOR², NR²R³, CONR²R³, SR², guanidino, alkynyl, and azido; or a pharmaceutically acceptable salt, solvate, or stereoisomer thereof. The compounds of the invention are trifluoroborate mimics of amino acids and are thus organotrifluoroborate mimics.

In another embodiment, A is alkylenyl. In another embodiment, A is C₁-C₅ alkylenyl. In yet another embodiment, A is methylenyl, ethylenyl, isobutylenyl, or isopentylenyl. In another embodiment, the BF₃ and NR¹R²R³⁺ moieties are attached to the same carbon of A.

In an another embodiment, the compound is of the Formula (II):

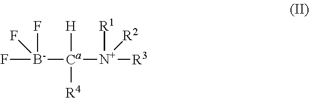

wherein C^{a} is in the R or S configuration; and R⁴ is H or alkyl, the alkyl optionally substituted with one or more substituents selected from halo, alkoxyl, cycloalkyl, heterocycloalkyl, aryl, heteroaryl, hydroxyl, COOR², NR²R³, CONR²R³, SR², guanidino, alkynyl, and azido, wherein each substituent cycloalkyl, heterocycloalkyl, aryl, and heteroaryl is optionally fused with one or more groups selected from cycloalkyl, heterocycloalkyl, aryl, and heteroaryl, wherein each substituent alkoxyl, cycloalkyl, heterocycloalkyl, aryl, and heteroaryl is optionally further substituted with one or more substituents selected from halo, alkyl, cycloalkyl, heterocycloalkyl, aryl, heteroaryl, hydroxyl, COOR², NR²R³, CONR²R³, SR², guanidino, alkynyl, and azido, and wherein substituent guanidino is optionally further substituted with one or more substituents selected from alkyl, cycloalkyl, heterocycloalkyl, aryl, and heteroaryl; wherein each of R¹, R², and R³ is independently H or alkyl, the alkyl optionally substituted with one or more substituents selected from halo, alkoxyl, cycloalkyl, heterocycloalkyl, aryl, heteroaryl, hydroxyl, COOR², NR²R³, CONR²R³, SR², guanidino, alkynyl, and azido; or R⁴ and R¹ together with the nitrogen of the NR¹R²R³⁺ moiety form a heterocycloalkyl or heteroaryl ring, wherein the ring is optionally substituted with one or more substituents selected from halo, alkyl, alkoxyl, cycloalkyl, heterocycloalkyl, aryl, heteroaryl, hydroxyl, COOR², NR²R³, CONR²R³, SR², guanidino, alkynyl, and azido; or a pharmaceutically acceptable salt, solvate, or stereoisomer thereof.

In another embodiment, the compound is

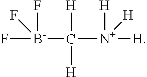

In another embodiment, the compound is one of the following:

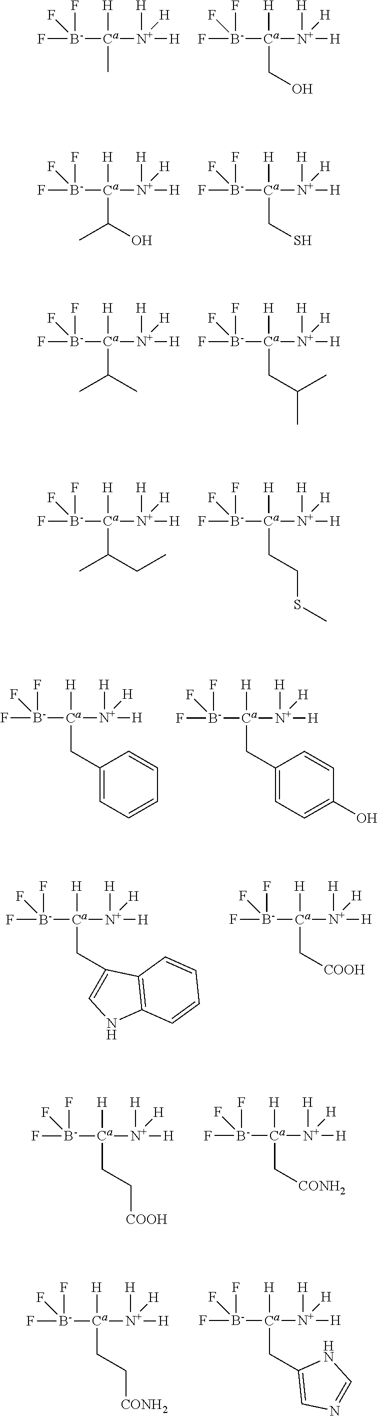

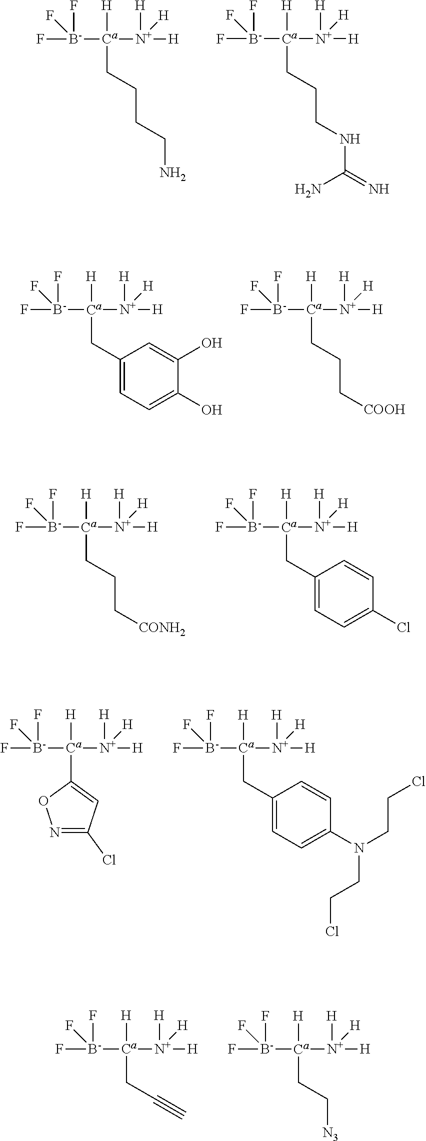

wherein C^{a} is in the R or S configuration.

In another embodiment, A and R¹ of Formula (I) or R⁴ and R¹ of Formula (II) together with the nitrogen of the NR¹R²R³⁺ moiety form a heterocycloalkyl or heteroaryl ring. In another embodiment, the compound is

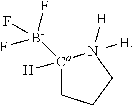

In yet another embodiment, the carbon designated as C^{a} is in the R configuration. In another embodiment, C^{a} is in the S configuration. Thus, the configurations of C^{a} giving D and L forms of, e.g., α-amino acids, are contemplated.

In another embodiment, at least one F is ¹⁸F. In another embodiment, more than one (e.g., 2 or 3) fluorines are ¹⁸F. In another embodiment, the compound, salt, solvate, or stereoisomer is one of the following:

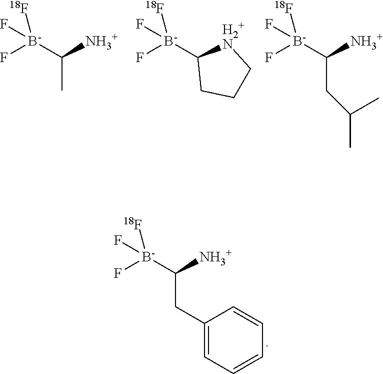

In some embodiments, the compound is one of the following:

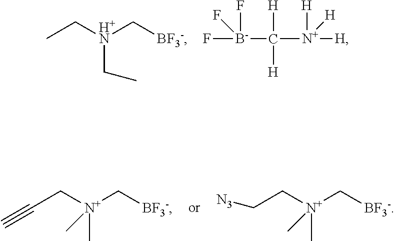

In some embodiments, the compound is not of the following:

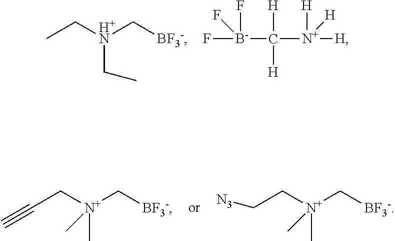

The compounds, salts, solvates, or stereoisomers of the present invention include all protonated forms. For example, the NR¹R²R³⁺ moiety of Formula (I) may be, for example, NH₂ or NH₃⁺. Also, for example, the BF₃⁻ moiety of Formula (I) may be BF₃⁻ or BF₃H. The protonation state may be modified based on the pH of, for example, any aqueous solvent used, which may depend on the use of buffering agents. In some embodiments, the compound is not any protonated form of

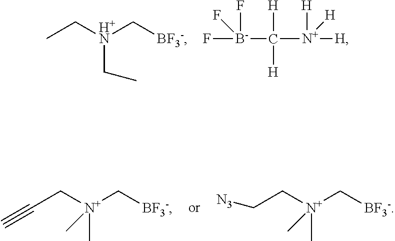

Table 1 below shows the pKa of the COOH group of the corresponding amino acid, the half-life (t_{1/2}) in phosphate buffer at pH 7.4, the rate of solvolysis (k), and the pK of the first fluorine of the BF₃⁻ moiety of five boramino acids. See also, Liu et al., Chem. Eur. J., 2015, 21:3924-8, incorporated herein by reference in its entirety.

TABLE 1

| | pka of corres. | | | |
|---|---|---|---|---|
| Name | COOH | t_{1/2}/min | k (solv)/min⁻¹ | pK_{B-F} |
| Ala-BF₃ | 2.35 | 3385 | 0.000204409 | 3.6895 |
| Leu-BF₃ | 2.33 | 3615 | 0.000191382 | 3.7181 |
| Phe-BF₃ | 2.2 | 5547 | 0.000124738 | 3.904 |
| Met-BF₃ | 2.13 | 6985 | 9.90604E−05 | 4.0041 |
| Pro-BF₃ | 1.95 | 12635 | 5.47646E−05 | 4.2615 |

Table 2 shows the major amino acid transporters (AATs) for various ¹⁸F-amino acids and boramino acids.

TABLE 2

| | Major | | |
|---|---|---|---|
| Name | AATs | ¹⁸F-AA | ¹⁸F-BAA |
| Ala | System A/ ASCT | N.A. | 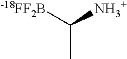 |
| Val | System L | N.A. | 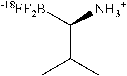 |
| Ile | System L | N.A. | 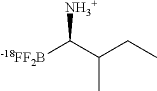 |
| Leu | System L | N.A. | 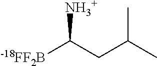 |
| Met | System L | N.A. | 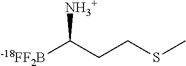 |
| Phe | System L | 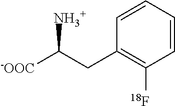 | 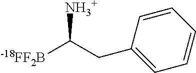 |
| Tyr | System L | 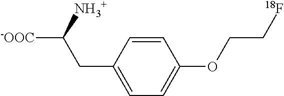 | 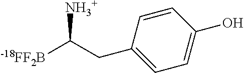 |
| Trp | System L | 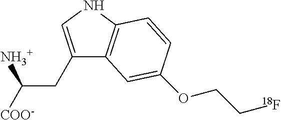 | 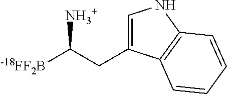 |
| Arg | System CAAT | N.A. | 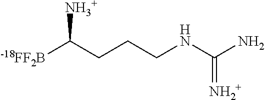 |
| His | System CAAT | N.A. | 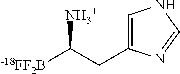 |
| Lys | System CAAT | N.A. | 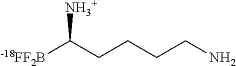 |
| Asp | System EAAT | N.A. | 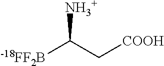 |
| Glu | System EAAT | 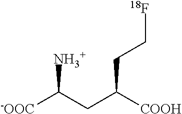 | 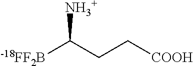 |
| Ser | System ASCT | N.A. | 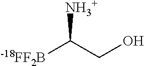 |
| Thr | System ASCT | N.A. | 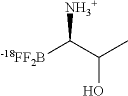 |
| Asn | System ATA | N.A. | 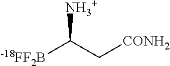 |
| Gln | System ATA | 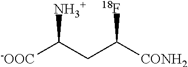 | 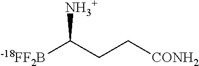 |
| Pro | System P | 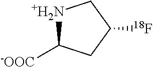 | 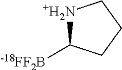 |
| Gly | System G | N.A. | 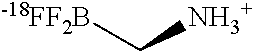 |
| Cys | System ASCT | N.A. | 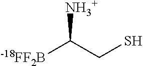 |

LAT-1 is the main channel for transport of most essential amino acids. LAT-1 demonstrates high affinity for the transportation of branched chain amino acids (Leu, Ile, and Val) as well the bulky amino acids (Phe, Trp, Tyr, Gln, Asn and Met). Leucine is transported by LAT-1 with unexpectedly high selectivity, where cellular uptake increases with increased LAT-1 expression. LAT-1 is over expressed on cancers and has been demonstrated to be an efficient target for the development of anti-cancer drugs. U87 glioma cells overexpress the system L transporter. In most cases, only LAT-1 is up-regulated on cancer cells, whereas LAT-2, LAT-3, and LAT-4 are found to be overexpressed on certain types of normal tissues, such as skeletal muscle, blood brain barrier, and fetal liver.

The term “amino acid” as used herein includes amino acids having the amino moiety and acid moiety attached to the same carbon, as in naturally occurring and non-naturally occurring alpha-amino acids, as well as attached to any of the different carbons along an alkyl chain with two or more carbon atoms, such as in, for example, beta-amino acids. Proline, although an imino acid, is considered one of the naturally occurring amino acids. The amino acid can be in the L- or D-forms, preferably the L-form.

In any of the embodiments above, the term “alkyl” implies a straight-chain or branched alkyl containing, for example, from 1 to 6 carbon atoms, e.g., from 1 to 4 carbon atoms. Examples of alkyl group include methyl, ethyl, n-propyl, isopropyl, n-butyl, sec-butyl, isobutyl, tert-butyl, n-pentyl, isopentyl, n-hexyl, and the like. This definition also applies wherever “alkyl” occurs as part of a group, such as, e.g., fluoro C₁-C₆ alkyl. The alkyl may be substituted or unsubstituted, as described herein. “Alkylenyl” is an alkyl radical attached to at least two groups, such as the BF₃ and NR¹R²R³⁺ moieties of Formula (I).

In any of the embodiments above, the term “cycloalkyl,” as used herein, means a cyclic alkyl moiety containing from, for example, 3 to 6 carbon atoms or from 5 to 6 carbon atoms. Examples of such moieties include cyclopropyl, cyclobutyl, cyclopentyl, cyclohexyl, and the like. The cycloalkyl may be substituted or unsubstituted, as described herein.

The term “heterocycloalkyl,” as used herein, means a stable, saturated, or partially unsaturated monocyclic, bicyclic, and spiro ring system containing 3 to 7 ring members of carbon atoms and other atoms selected from nitrogen, sulfur, and/or oxygen. In an aspect, a heterocycloalkyl is a 5, 6, or 7-membered monocyclic ring and contains one, two, or three heteroatoms selected from nitrogen, oxygen, and sulfur. The heterocycloalkyl may be attached to the parent structure through a carbon atom or through any heteroatom of the heterocycloalkyl that results in a stable structure. Examples of such heterocycloalkyl rings are isoxazolyl, thiazolinyl, imidazolidinyl, piperazinyl, homopiperazinyl, pyrrolinyl, pyrrolidinyl, pyrazolyl, pyranyl, piperidyl, oxazolyl, and morpholinyl. The heterocycloalkyl may be substituted or unsubstituted, as described herein.

In any of the embodiments above, the term “hydroxyl” refers to the group —OH. In any of the embodiments, the term “guanidino” refers to the group —NH—(C═NH)—NH₂.

In any of the embodiments above, the terms “alkoxyl” and “aryloxyl” refer to linear or branched alkyl and aryl groups that are attached to a divalent oxygen. The alkyl and aryl groups are the same as described herein.

In any of the embodiments above, the term “halo” refers to a halogen selected from fluorine, chlorine, bromine, and iodine.

In any of the embodiments above, the term “aryl” refers to a mono, bi, or tricyclic carbocyclic ring system that may have one, two, or three aromatic rings, for example, phenyl, naphthyl, anthracenyl, or biphenyl. The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2 π electrons, according to Hückel's Rule, wherein n=1, 2, or 3. The aryl may be substituted or unsubstituted, as described herein.

In any of the embodiments above, the term “heteroaryl” refers to an aryl as defined above in which at least one, preferably 1 or 2, of the carbon atoms of the aromatic carbocyclic ring is replaced by N, O or S atoms. Examples of heteroaryl include pyridyl, furanyl, pyrrolyl, quinolinyl, thiophenyl, indolyl, imidazolyl and the like.

In other aspects, any substituent that is not hydrogen (e.g., C₁-C₆ alkyl, C₂-C₆ alkenyl, C₃-C₆ cycloalkyl, or aryl) may be an optionally substituted moiety. The substituted moiety typically comprises at least one substituent (e.g., 1, 2, 3, 4, 5, 6, etc.) in any suitable position (e.g., 1-, 2-, 3-, 4-, 5-, or 6-position, etc.). When an aryl group is substituted with a substituent, e.g., halo, amino, alkyl, OH, alkoxy, cyano, nitro, and others, the aromatic ring hydrogen is replaced with the substituent and this may take place in any of the available hydrogens, e.g., 2, 3, 4, 5, and/or 6-position wherein the 1-position is the point of attachment of the aryl group in the compounds, salts, solvates, or stereoisomers of the present invention. Suitable substituents include, e.g., halo, alkyl, alkenyl, alkynyl, hydroxy, nitro, cyano, amino, alkylamino, alkoxy, aryloxy, aralkoxy, carboxyl, carboxyalkyl, carboxyalkyloxy, amido, alkylamido, haloalkylamido, aryl, heteroaryl, and heterocycloalkyl. In some instances, the substituent is at least one alkyl, halo, and/or haloalkyl (e.g., 1 or 2).

In any of the embodiments above, whenever a range of the number of atoms in a structure is indicated (e.g., a C₁₋₆, or C₁₋₄ alkyl, C₃-C₆ cycloalkyl, etc.), it is specifically contemplated that any sub-range or individual number of carbon atoms falling within the indicated range also may be used. Thus, for instance, the recitation of a range of 1-6 carbon atoms (e.g., C₁-C₆), 1-4 carbon atoms (e.g., C₁-C₄), 1-3 carbon atoms (e.g., C₁-C₃), or 2-6 carbon atoms (e.g., C₂-C₆) as used with respect to any chemical group (e.g., alkyl, cycloalkyl, etc.) referenced herein encompasses and specifically describes 1, 2, 3, 4, 5, and/or 6 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 2-3 carbon atoms, 2-4 carbon atoms, 2-5 carbon atoms, 2-6 carbon atoms, 3-4 carbon atoms, 3-5 carbon atoms, 3-6 carbon atoms, 4-5 carbon atoms, 4-6 carbon atoms, etc., as appropriate).

A salt of a compound is a biologically acceptable salt, which is generally non-toxic, and is exemplified by salts with base or acid addition salts, inclusive of salts with inorganic base such as alkali metal salt (e.g., a sodium salt, a potassium salt), alkaline earth metal salt (e.g., calcium salt, magnesium salt), ammonium salt, salts with organic base such as organic amine salt (e.g., triethylamine salt, diisopropylethylamine salt, pyridine salt, picoline salt, ethanolamine salt, diethanolamine salt, triethanolamine salt, dicyclohexylamine salt, N, N′-dibenzylethylenediamine salt), inorganic acid salt (e.g., hydrochloride, hydrobromide, sulfate, phosphate), organic carboxylic or sulfonic acid salt (e.g., formate, acetate, trifluoroacetate, maleate, tartrate, fumarate, methanesulfonate, benzenesulfonate, toluenesulfonate), salt with basic or acid amino acid (e.g., arginine, aspartic acid, glutamic acid), and the like. In any of the embodiments above, the term “salt” encompasses “pharmaceutically acceptable salt.” Lists of suitable pharmaceutical salts are found in, for example, Remington's Pharmaceutical Sciences, 18th ed., Mack Publishing Company, Easton, Pa., 1990, p. 1445, and Journal of Pharmaceutical Science, 66, 2-19 (1977). For example, they may be a salt of an alkali metal (e.g., sodium or potassium), alkaline earth metal (e.g., calcium), or ammonium of salt.

Salts formed from free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

It is further understood that the compounds described herein may form solvates, or exist in a substantially uncomplexed form, such as the anhydrous form. Those of skill in the art appreciate that many organic compounds can form complexes with solvents in which they are reacted or from which they are precipitated or crystallized. These complexes are known as “solvates.” A solvate is a molecule consisting of a complex made up of solute molecules and solvent molecules resulting from the solution. For example, a complex with water is known as a “hydrate.” Solvates as defined herein may be crystalline or non-crystalline, such as amorphous, and may be formed by any suitable method, including, but not limited to reaction, precipitation, or crystallization. Solvates of the compounds, salts, and stereoisomers described herein, including pharmaceutically acceptable solvates, are within the scope of the invention.

It will also be appreciated by those of skill in the art that many organic compounds can exist in more than one crystalline form (polymorphic forms). For example, crystalline form may vary from solvate to solvate. Thus, all crystalline forms of the compounds, salts, solvates, and stereoisomers described herein are within the scope of the present invention. Pharmaceutically acceptable solvates include hydrates, alcoholates such as methanolates and ethanolates, acetonitrilates and the like.

A compound can have stereoisomers based on asymmetric carbon atoms and double bonds, such as optical isomers, geometric isomers, and the like, all of which and mixtures thereof are also encompassed in the present invention.

The methods described herein comprise administering a compound, salt, solvate, or stereoisomer of Formula (I) in the form of a composition. In particular, a composition will comprise at least one compound, salt, solvate, or stereoisomer of Formula (I) and a pharmaceutically acceptable carrier. The pharmaceutically acceptable excipients described herein, for example, vehicles, adjuvants, carriers or diluents, are well-known to those who are skilled in the art and are readily available to the public. Typically, the pharmaceutically acceptable carrier is one that is chemically inert to the active compound, salt, solvate, or stereoisomer and one that has no detrimental side effects or toxicity under the conditions of use.

The compositions may be administered as oral, sublingual, transdermal, subcutaneous, topical, absorption through epithelial or mucocutaneous linings, intravenous, intranasal, intraarterial, intramuscular, intratumoral, peritumoral, interperitoneal, intrathecal, rectal, vaginal, or aerosol formulations. In some aspects, the composition is administered orally or intravenously.

In accordance with any of the embodiments, a compound, salt, solvate, or stereoisomer of Formula (I) may be administered orally to a subject in need thereof. Formulations suitable for oral administration may consist of (a) liquid solutions, such as an effective amount of the compound, salt, solvate, or stereoisomer dissolved in diluents, such as water, saline, or orange juice and include an additive, such as cyclodextrin (e.g., α-, β-, or -γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms may be of the ordinary hard- or soft-shelled gelatin type containing, for example, surfactants, lubricants, and inert fillers, such as lactose, sucrose, calcium phosphate, and cornstarch. Tablet forms may include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms may comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which may contain anti-oxidants, buffers, bacteriostats, and solutes that render the formulation isotonic with the blood of the intended recipient, and aqueous and non-aqueous sterile suspensions that may include suspending agents, solubilizers, thickening agents, stabilizers, and preservatives. The compound, salt, solvate, or stereoisomer of Formula (I) may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which may be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (e) mixtures thereof.

The parenteral formulations will typically contain from about 0.5 to about 25% by weight of the compound, salt, solvate, or stereoisomer of Formula (I) in solution. Suitable preservatives and buffers may be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations may be presented in unit-dose or multi-dose sealed containers, such as ampoules and vials, and may be stored in a freeze-dried (lyophilized) condition requiring only the addition of the sterile liquid carrier, for example, water, for injections, immediately prior to use. Extemporaneous injection solutions and suspensions may be prepared from sterile powders, granules, and tablets of the kind previously described.

The compound, salt, solvate, or stereoisomer of Formula (I) may be made into an injectable formulation. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), creams, pastes, lotions and gels. Topical administration includes application to the oral mucosa, which includes the oral cavity, oral epithelium, palate, gingival, and the nasal mucosa. In some embodiments, the composition contains at least one active component and a suitable vehicle or carrier. It may also contain other components, such as an anti-irritant. The carrier may be a liquid, solid or semi-solid. In embodiments, the composition is an aqueous solution, such as a mouthwash. Alternatively, the composition may be a dispersion, emulsion, gel, lotion or cream vehicle for the various components. In one embodiment, the primary vehicle is water or a biocompatible solvent that is substantially neutral or that has been rendered substantially neutral. The liquid vehicle may include other materials, such as buffers, alcohols, glycerin, and mineral oils with various emulsifiers or dispersing agents as known in the art to obtain the desired pH, consistency and viscosity. It is possible that the compositions may be produced as solids, such as powders or granules. The solids may be applied directly or dissolved in water or a biocompatible solvent prior to use to form a solution that is substantially neutral or that has been rendered substantially neutral and that may then be applied to the target site. In embodiments of the invention, the vehicle for topical application to the skin may include water, buffered solutions, various alcohols, glycols such as glycerin, lipid materials such as fatty acids, mineral oils, phosphoglycerides, collagen, gelatin and silicone based materials.

The compound, salt, solvate, or stereoisomer of Formula (I), alone or in combination with other suitable components, may be made into aerosol formulations to be administered via inhalation. These aerosol formulations may be placed into pressurized acceptable propellants, such as dichlorodifluoromethane, propane, nitrogen, and the like. They also may be formulated as pharmaceuticals for non-pressured preparations, such as in a nebulizer or an atomizer.

It will be appreciated by one of ordinary skill in the art that, in addition to the aforedescribed compositions, a compound, salt, solvate, or stereoisomer of the invention may be formulated as inclusion complexes, such as cyclodextrin inclusion complexes, or liposomes. Liposomes may serve to target a compound, salt, solvate, or stereoisomer of the invention to a particular tissue, such as lymphoid tissue or cancerous hepatic cells. Liposomes may also be used to increase the half-life of a compound, salt, solvate, or stereoisomer of the invention. Many methods are available for preparing liposomes, as described in, for example, Szoka et al., Ann. Rev. Biophys. Bioeng. 1980, 9, 467 and U.S. Pat. Nos. 4,235,871, 4,501,728, 4,837,028, and 5,019,369.

The dose administered to the mammal, particularly human and other mammals, in accordance with the present invention should be sufficient to affect the desired response, e.g., a favorable PET imaging signal-to-noise ratio. One skilled in the art will recognize that dosage will depend upon a variety of factors, including the age, condition or disease state, predisposition to disease, genetic defect or defects, and body weight of the mammal. The size of the dose will also be determined by the route, timing and frequency of administration as well as the existence, nature, and extent of any adverse side-effects that might accompany the administration of a particular compound, salt, solvate, or stereoisomer and the desired effect. It will be appreciated by one of skill in the art that various conditions may require prolonged or multiple administrations.

The inventive methods comprise administering an effective amount of a compound, salt, solvate, or stereoisomer of Formula (I). An “effective amount” means an amount sufficient to, e.g., provide a favorable PET imaging signal-to-noise ratio. The signal to noise ratio may be to any suitable degree, e.g., when the ratio of signal-to-noise is greater than 1.5.

Effective amounts may vary depending upon the biological effect desired in the individual and/or the specific characteristics of the compound, salt, solvate, or stereoisomer of Formula (I), and the individual (e.g., a 70 kg patient on average). In this respect, any suitable dose of the compound, salt, solvate, or stereoisomer of Formula (I) may be administered to the mammal (e.g., human). Various general considerations taken into account in determining the “effective amount” are known to those of skill in the art. The dose of the compound, salt, solvate, or stereoisomer of Formula (I) desirably comprises about 0.001 mg per kilogram (kg) of the body weight of the mammal (mg/kg) to about 400 mg/kg. The minimum dose is any suitable amount, such as about 0.001 mg/kg, about 0.005 mg/kg, about 0.0075 mg/kg, about 0.01 mg/kg, about 0.05 mg/kg, about 0.075 mg/kg, about 0.1 mg/kg, about 0.15 mg/kg, about 0.2 mg/kg, about 0.4 mg/kg, about 0.75 mg/kg, about 1 mg/kg, about 2 mg/kg, about 3 mg/kg, about 5 mg/kg, about 10 mg/kg, about 15 mg/kg, about 20 mg/kg, about 30 mg/kg, about 50 mg/kg, about 60 mg/kg, about 75 mg/kg, about 100 mg/kg, about 150 mg/kg, about 175 mg/kg, about 200 mg/kg, about 250 mg/kg, about 275 mg/kg, or about 300 mg/kg). The maximum dose is any suitable amount, such as about 350 mg/mg, about 300 mg/kg, about 275 mg/kg, about 250 mg/kg, about 200 mg/kg, about 175 mg/kg, about 150 mg/kg, about 100 mg/kg, about 75 mg/kg, about 60 mg/kg, about 50 mg/kg, about 30 mg/kg, about 20 mg/kg, about 15 mg/kg, about 10 mg/kg, about 5 mg/kg, about 3 mg/kg, about 2 mg/kg, about 1 mg/kg, about 0.75 mg/kg, about 0.4 mg/kg, or about 0.2 mg/kg). Any two of the foregoing minimum and maximum doses may be used to define a close-ended range or may be used singly to define an open-ended range.

The invention also provides a method of imaging cancer in a mammal comprising administering to the mammal an effective amount of a compound, salt, solvate, or stereoisomer of Formula (I). Suitable cancers include cancers of the head and neck, eye, skin, mouth, throat, esophagus, chest, bone, lung, colon, sigmoid, rectum, stomach, prostate, breast, ovaries, kidney, liver, pancreas, brain, intestine, heart, or adrenals. More particularly, cancers include solid tumor, sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's sarcoma (tumor), leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, retinoblastoma, a blood-borne tumor, acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acutenonlymphocyctic leukemia, acute undifferentiated leukemia, chronic myelocytic leukemia, chronic lymphocytic leukemia, hairy cell leukemia, or multiple myeloma. See, e.g., Harrison's Principles of Internal Medicine, Eugene Braunwald et al., eds., pp. 491 762 (15th ed. 2001). Preferred are glioma, prostate cancer, and pancreas cancer.

A compound, salt, solvate, or stereoisomer of Formula (I) may be administered, simultaneously or sequentially or cyclically, in a coordinate protocol with one or more secondary or adjunctive agents. Thus, in certain embodiments compound, salt, solvate, or stereoisomer of Formula (I) is administered coordinately with a different agent, or any other secondary or adjunctive agent, utilizing separate formulations or a combinatorial formulation as described above (i.e., comprising both compound, salt, solvate, or stereoisomer of Formula (I) and another agent). This coordinate administration may be done simultaneously or sequentially in either order, and there may be a time period while only one or both (or all) active agents individually and/or collectively exert their biological activities.

For purposes of the present invention, the subject or individual typically is a mammal. Mammals include, but are not limited to, the order Rodentia, such as mice, and the order Logomorpha, such as rabbits. In some aspects, the mammals are from the order Carnivora, including Felines (cats) and Canines (dogs), Artiodactyla, including Bovines (cows) and Swines (pigs) or of the order Perssodactyla, including Equines (horses). In some aspects, the mammals are of the order Primates, Ceboids, or Simioids (monkeys) or of the order Anthropoids (humans and apes). In embodiments of the invention, the mammal is a human.

The compounds, salts, solvates, or stereoisomers described herein may be prepared by any suitable synthetic methodology. For example, the Schemes 1 and 2 present synthetic routes with high enantiopurity.

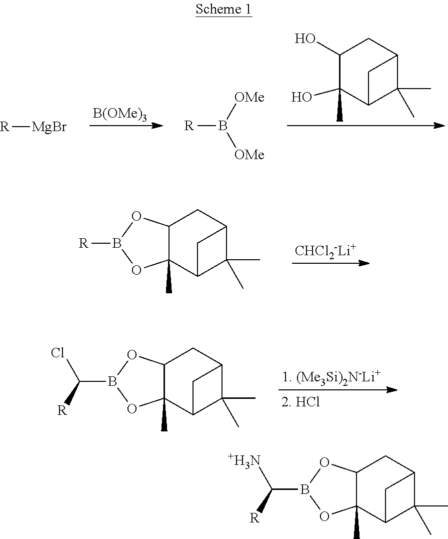

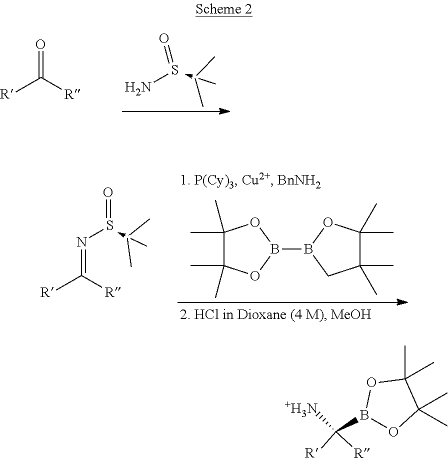

Scheme 3 presents an exemplary method of fluorination.

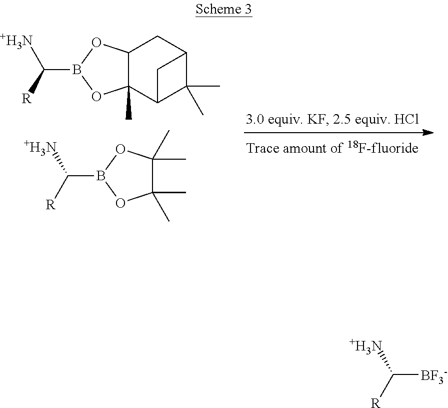

The fluorination of scheme 3 may be achieved, e.g., using KF or KHF₂.

One of ordinary skill in the art understands how to perform the above synthetic schemes using suitable conditions to produce the products shown.

An exemplary process to produce Phe-BF₃ is shown in Scheme 4 below.

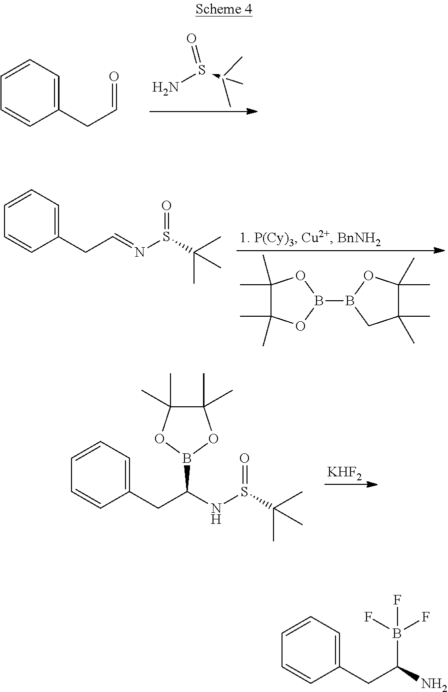

As another example, an amino acid derivative may be produced wherein a boron moiety, e.g., Bpin (boronic acid pinacol ester), is in the place of the carboxyl group. The boron moiety group may then be converted to a BF₃⁻ moiety, e.g., through fluorination, e.g., as shown in Scheme 3 above. Also, for example, commercially available compounds may be converted to compounds described herein. For example, leucine, proline, and alanine are commercially available from Advanced ChemBlock, Inc. (Burlingame, Calif., USA) as the boronic ester ((R)-BoroAla-(+)-pinanediol, Catalog #: 10178; (R)-BoroPro-(+)-Pinanediol-HCl, Catalog #: 10090; (R)-BoroLeu-(+)-pinanediol, Catalog #: 10181) such that they may be converted to the BAA through fluorination, e.g., as in Scheme 3. Also see, e.g., Kelly et al., Tetrahedron, 1993, 49:1009-16, for synthesis of a proline boronate ester and Thomassen, Synthesis of β-substituted β-aminoboronates, KJE-3900 Master's Thesis in Chemistry, University of Tromsø, May 2013 (available at www.ub.uit.no/munin/), each incorporated by reference herein in its entirety. Boronic esters may be used, for example, those of alpha-, beta-, gamma-, delta-, omega-amino acids and the like. See, e.g., Kinder et al., J. Med. Chem., 1990, 33:819-23; Kinder et al., J. Org. Chem., 1987, 52:2452-4; Brnardic et al., Bioorg. Med. Chem. Lett., 2007, 17:5989-4; Jego et al., J. Organomet. Chem., 1992, 435:1-8; each incorporated by reference herein in its entirety.

In another embodiment, the present invention provides a process for the preparation of a compound or a pharmaceutically acceptable salt, solvate, or stereoisomer thereof, wherein the compound is of the Formula (I), the process comprising reacting a compound of Formula (III)

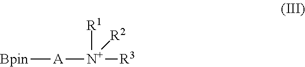

with fluorine under suitable conditions to produce a compound of Formula (I).

In another embodiment, the present invention provides a process for the preparation of a compound, salt, solvate, or stereoisomer described herein, the process comprising (i) boronating a Grignard reagent comprising R⁴ under suitable conditions, e.g., R⁴ under −78° C. in anhydrous tetrahydrofuran, to produce a compound of R⁴—B(OMe)₂; (ii) reacting the R⁴—B(OMe)₂ with

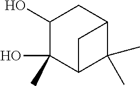

under suitable conditions to produce a compound of (P1):

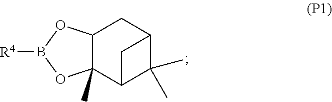

(iii) reacting (P1) with Li⁺CHCl₂⁻ under suitable conditions, e.g., under −78° C. in anhydrous tetrahydrofuran, to produce a compound of (P2):

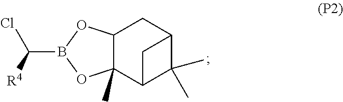

and
(iv) replacing the chlorine of (P2) with amino, the replacement optionally made by reacting (P2) with (Me₃Si)₂N⁻Li⁺ and HCl under suitable conditions to produce a compound of (P3):

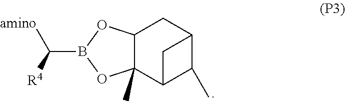

In another embodiment, the present invention provides a process for the preparation of a compound, salt, solvate, or stereoisomer described herein, the process comprising (i) reacting R⁴CHO with H₂NSOC(CH₃)₃ under suitable conditions to produce a compound of R⁴CH═NSOC(CH₃)₃; (ii) boronating R⁴CH═NSOC(CH₃)₃ under suitable conditions to produce a compound of (P4):

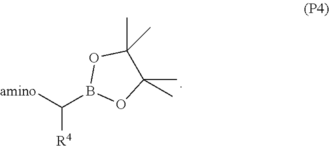

In another embodiment, the present invention provides a process for the preparation of a compound, salt, solvate, or stereoisomer described herein, the process comprising fluorinating the boron moiety of

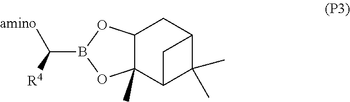

under suitable conditions to produce a compound of

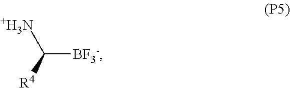

the fluorination optionally performed by reacting the (P3) with KF or KHF₂ and HCl.

In another embodiment, the present invention provides a process for the preparation of a compound, salt, solvate, or stereoisomer described herein, the process comprising fluorinating the boron moiety of

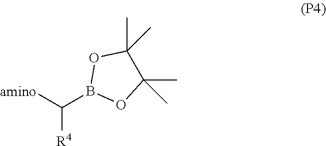

under suitable conditions, e.g., acidic conditions, to produce a compound of

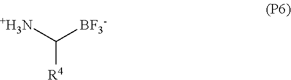

the fluorination optionally performed by reacting the (P4) with KF or KHF₂ and HCl.

In another embodiment, the present invention provides a process for the preparation of a salt, solvate, or stereoisomer described herein, the process comprising exchanging ¹⁹F for ¹⁸F under suitable conditions, e.g., acidic conditions, the exchange optionally made by incubating the boramino acid with ¹⁸F-fluoride water.

One embodiment includes labeling the compounds, salts, solvates, or stereoisomers with ¹⁸F using one step. Advantages of one step labeling include utilization of aqueous solution without the need for tedious azeotropic drying. Another advantage includes the relative ease of purification utilizing solid-phase extraction without the need for HPLC. Also, the radiosynthesis/purification time is short (e.g., within 30 min), and there are good radiochemical yields (˜60%, non-decay-corrected), high purity (>99%), and specific activity (radioactivity per unit of compound) or ˜37 GBq/μmol. This ¹⁸F-labeling strategy was successfully applied for trifluoroborate conjugates, including: ¹⁸F-AmBF₃-TATE, ¹⁸F-AmBF₃-Rhodamine-BisRGD, ¹⁸F-AmBF₃-Bradykinin, and others (see, e.g., Liu et al., J. Nucl. Med., 2014, 55:1499-505; Liu et al., Angew Chem. Int. Ed. Engl., 2014, 53:11876-80; Liu et al., Bioconjug. Chem., 2014, 25:1951-62, each incorporated by reference in its entirety). In addition, this radiochemistry does not necessitate the preparation of complex organometallic precursors and may be performed with commercially available reagents in a reaction vessel exposed to air.

In yet another embodiment, the present invention provides a method of imaging a tumor within a subject comprising administering to the subject an effective amount of a compound, salt, solvate, or stereoisomer with at least one fluorine being ¹⁸F, or a composition thereof, as described herein, and utilizing positron emission tomography to take an image of the tumor within the subject. In another embodiment, the method further comprises taking more than one image of the tumor, wherein the images are taken at different times, and measuring the size of the tumor on each image. Using such a method, the effectiveness of potential anti-cancer drugs may be evaluated, e.g., by following tumor size and comparing to drug identity, drug dosage, treatment duration, etc. Compared with traditional amino acid drugs, boramino acids can be a potent candidate to provide better specificity and efficacy for cancer treatment. BAAs are more robust against metabolism and show less uptake in healthy tissue but high uptake in tumor, as tumors overly expresses amino acid transporters.

Non-invasively differentiating tumor from inflammation is a long-standing challenge for clinical cancer diagnosis. PET with ¹⁸F-FDG (fluordeoxyglucose) is the standard non-invasive technique for cancer imaging. However, because ¹⁸F-FDG accumulation in tumor cells depends on glucose metabolism, ¹⁸F-FDG accumulates in any tissue with high glucose consumption. ¹⁸F-FDG also shows essentially non-specific uptake in the brain and may be observed in a variety of healthy tissues and ones affected by various non-neoplastic pathologic conditions, such as acute and chronic inflammation and infection. In a recent study, when ¹⁸F-FDG was used to evaluate the malignancy of lung nodules, the false positive rate of misidentifying non-tumorous tissue as a tumor was found to be nearly 40%; and for patients who have surgery or biopsy, 35% were eventually found to have only benign disease. This high false positive rate often misleads physicians, who may give improper treatment or management strategies. This may result in higher clinical cost and sometimes in missing the best time point for treatment.

BAAs can afford higher uptake in human cancer xenografts than naturally occurring AAs but do not show notable uptake in inflammation. In an embodiment of the invention, BAAs may be used in the development of next generation cancer imaging probes as well as chemotherapeutic agents by replacing its side-chain with other cellular toxic moieties.

The results of the inventive tracers are particularly impressive compared to previously established amino acid tracers. Based on dynamic PET scans, after blood circulation, radiolabeled amino acids quickly distribute to the entire body in a very short period. To give a high tumor-to-background ratio, the tracer should be capable to be cleared from the non-tumor tissue and re-enriched in the transporter upregulated cancer cells. In this case, metabolic stability is rather important. Some naturally occurring amino acids cannot be cleared from non-tumor tissue because of their participation in protein synthesis. In contrast, boramino acids are not capable of forming an amide bond with regular amino acids due to the trifluoroborate group. As BAAs will not participate in protein synthesis, they may have fast clearance from tumors.

In another embodiment, the present invention provides a method of imaging tumor uptake, the method comprising identifying an amino acid having at least one COO⁻ moiety, generating a boramino acid mimic, wherein the boramino acid mimic has the same structure as the amino acid except a COO⁻ moiety of the amino acid is replaced with a BF₃⁻ moiety in the boramino acid mimic, wherein at least one fluorine is ¹⁸F, administering to a subject with a tumor an effective amount of the boramino acid mimic, and utilizing positron emission tomography to take an image of the tumor within the subject. In another embodiment, this method further comprises taking more than one image of the tumor, wherein the images are taken at different times, and measuring the size of the tumor on each image.

In another embodiment, the present invention also provides a method of treating a tumor in a subject, the method comprising administering to the subject an effective amount of a compound, salt, solvate, stereoisomer, or composition thereof as described herein and irradiating the subject with neutrons.

Boron neutron capture therapy (BNCT) is a non-invasive method of treating tumors, including tumors that are deep within the body and those that are inoperable. BNCT utilizes a boron-containing capture agent that is administered to a subject and localizes to tumors within the subject. The boron has a high propensity to capture slow neutrons, whereas other elements such as H, O, and N do not. After administration of the capture agent to the subject, the subject is irradiated with neutrons, e.g., epithermal neutrons, which penetrate tissue and are absorbed by the capture agent. Upon absorption, the capture agent emits high-energy particles that destroy the cells containing the capture agent. As BAAs are concentrated in tumor tissue, the BAAs of the present invention may be suitable capture agents in BNCT. BNCT is described in Moss, Applied Radiation and Isotopes, 88: 2-11 (2014); Barth et al. Clinical Cancer Research, 11: 3987-4002 (2005); and Barth et al. Radiation Oncology, 7: 146 (2012), each of which is incorporated herein by reference.

The following includes certain aspects of the invention.
1. A compound of the Formula (I):

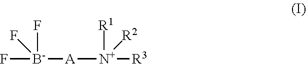

wherein
A is optionally substituted alkylenyl, wherein the substituents are selected from halo, alkoxyl, cycloalkyl, heterocycloalkyl, aryl, heteroaryl, hydroxyl, COOR², NR²R³, CONR²R³, SR², guanidino, alkynyl, and azido,
wherein each substituent cycloalkyl, heterocycloalkyl, aryl, and heteroaryl is optionally fused with one or more groups selected from cycloalkyl, heterocycloalkyl, aryl, and heteroaryl,
wherein each substituent alkoxyl, cycloalkyl, heterocycloalkyl, aryl, and heteroaryl is optionally further substituted with one or more substituents selected from halo, alkyl, cycloalkyl, heterocycloalkyl, aryl, heteroaryl, hydroxyl, COOR², NR²R³, CONR²R³, SR², guanidino, alkynyl, and azido,
wherein substituent guanidino is optionally further substituted with one or more substituents selected from alkyl, cycloalkyl, heterocycloalkyl, aryl, and heteroaryl,
wherein each of R¹, R², and R³ is independently H or alkyl, the alkyl optionally substituted with one or more substituents selected from halo, alkoxyl, cycloalkyl, heterocycloalkyl, aryl, heteroaryl, hydroxyl, COOR², NR²R³, CONR²R³, SR², guanidino, alkynyl, and azido;
or
A and R¹ together with the nitrogen of the NR¹R²R³⁺ moiety form a heterocycloalkyl or heteroaryl ring, wherein the ring is optionally substituted with one or more substituents selected from halo, alkyl, alkoxyl, cycloalkyl, heterocycloalkyl, aryl, heteroaryl, hydroxyl, COOR², NR²R³, CONR²R³, SR², guanidino, alkynyl, and azido;
or a pharmaceutically acceptable salt, solvate, or stereoisomer thereof.
2. The compound, salt, solvate, or stereoisomer of aspect 1, wherein A is alkylenyl.
3. The compound, salt, solvate, or stereoisomer of aspect 2, wherein A is C₁-C₅ alkylenyl.
4. The compound, salt, solvate, or stereoisomer of aspect 2 or 3, wherein A is methylenyl, ethylenyl, isobutylenyl, or isopentylenyl.
5. The compound, salt, solvate, or stereoisomer of any one of aspects 2-4, wherein the BF₃ and NR¹R²R³⁺ moieties are attached to the same carbon of A.
6. The compound, salt, solvate, or stereoisomer of any one of aspects 1-5, wherein the compound is of the Formula (II):

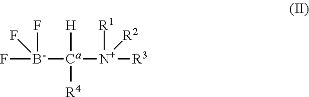

wherein
C^{a} is in the R or S configuration; and
R⁴ is H or alkyl, the alkyl optionally substituted with one or more substituents selected from halo, alkoxyl, cycloalkyl, heterocycloalkyl, aryl, heteroaryl, hydroxyl, COOR², NR²R³, CONR²R³, SR², guanidino, alkynyl, and azido,
wherein each substituent cycloalkyl, heterocycloalkyl, aryl, and heteroaryl is optionally fused with one or more groups selected from cycloalkyl, heterocycloalkyl, aryl, and heteroaryl, wherein each substituent alkoxyl, cycloalkyl, heterocycloalkyl, aryl, and heteroaryl is optionally further substituted with one or more substituents selected from halo, alkyl, cycloalkyl, heterocycloalkyl, aryl, heteroaryl, hydroxyl, COOR², NR²R³, CONR²R³, SR², guanidino, alkynyl, and azido, and
wherein substituent guanidino is optionally further substituted with one or more substituents selected from alkyl, cycloalkyl, heterocycloalkyl, aryl, and heteroaryl; or
R⁴ and R¹ together with the nitrogen of the NR¹R²R³⁺ moiety form a heterocycloalkyl or heteroaryl ring, wherein the ring is optionally substituted with one or more substituents selected from halo, alkyl, alkoxyl, cycloalkyl, heterocycloalkyl, aryl, heteroaryl, hydroxyl, COOR², NR²R³, CONR²R³, SR², guanidino, alkynyl, and azido.
7. The compound, salt, solvate, or stereoisomer of any one of aspects 1-6, wherein the compound is one of the following:

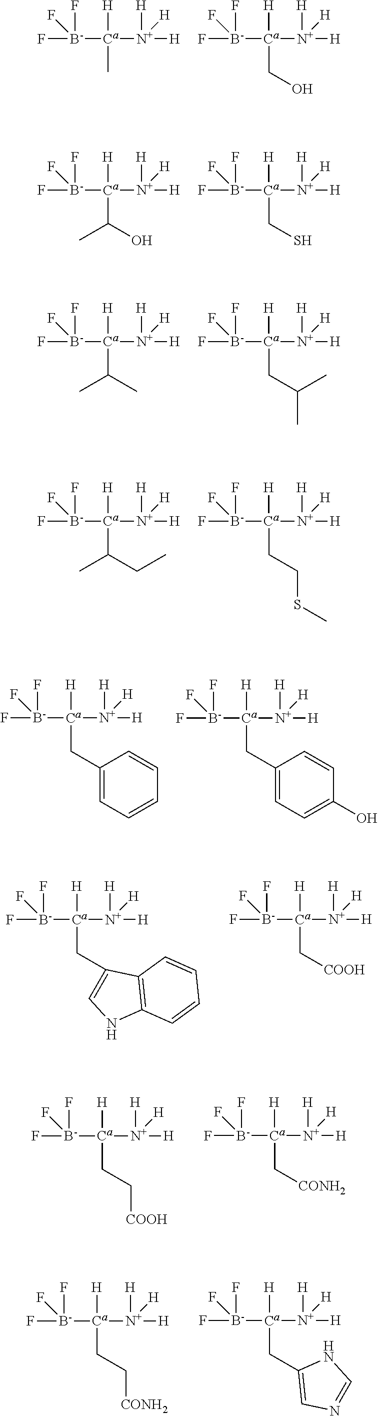

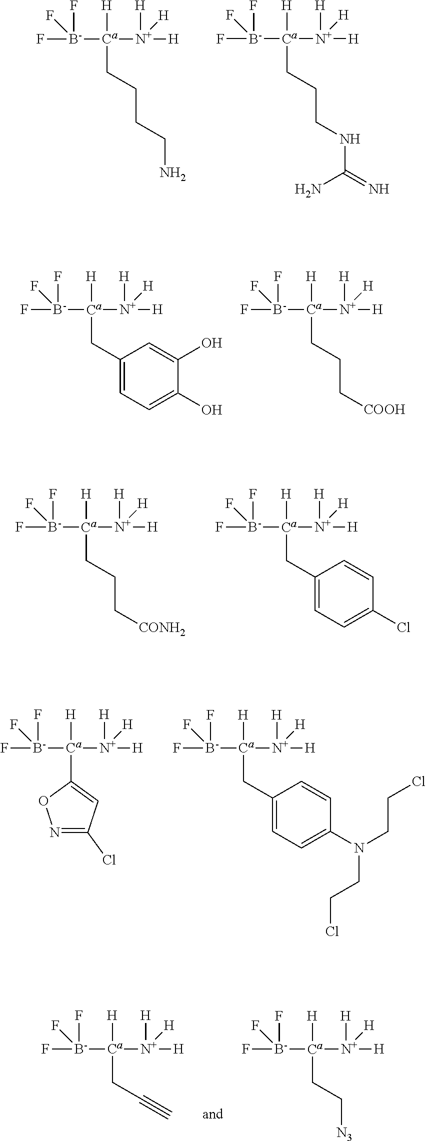

wherein C^{a} is in the R or S configuration.
8. The compound, salt, solvate, or stereoisomer of aspect 1, wherein A and R¹ together with the nitrogen of the NR¹R²R³⁺ moiety form a heterocycloalkyl or heteroaryl ring.
9. The compound, salt, solvate, or stereoisomer of aspect 6, wherein R⁴ and R¹ together with the nitrogen of the NR¹R²R³⁺ moiety form a heterocycloalkyl or heteroaryl ring.
10. The compound, salt, solvate, or stereoisomer of aspect 8 or 9, wherein the compound is

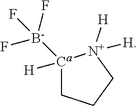

11. The compound, salt, solvate, or stereoisomer of aspect 7 or 10, wherein C^{a} is in the R configuration.
12. The compound, salt, solvate, or stereoisomer of aspect 7 or 10, wherein C^{a} is in the S configuration.
13. The compound, salt, solvate, or stereoisomer of any one of aspects 1-12, wherein at least one F is ¹⁸F.
14. The compound, salt, solvate, or stereoisomer of any one of aspects 1-13, wherein more than one F is ¹⁸F.
15. The compound, salt, solvate, or stereoisomer of aspects 1, wherein the compound is one of the following:

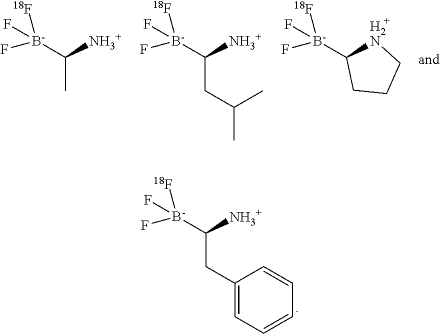

16. A composition comprising a compound, salt, solvate, or stereoisomer of any one of aspects 1-15 and a pharmaceutically acceptable carrier.
17. A method of imaging a tumor within a subject, the method comprising administering to the subject an effective amount of a compound, salt, solvate, or stereoisomer of aspect 14 or 15, or a composition of aspect 16 wherein the compound, salt, solvate, or stereoisomer contains at least one ¹⁸F, and utilizing positron emission tomography to take an image of the tumor within the subject.
18. The method of aspect 17, wherein the subject is a mammal.
19. The method of aspect 18, wherein the mammal is a human.
20. The method of any one of aspects 17-19, wherein the method further comprises taking more than one image of the tumor, wherein the images are taken at different times, and measuring the size of the tumor on each image.
21. A method of imaging tumor uptake, the method comprising: identifying an amino acid having at least one COO⁻ moiety, generating a boramino acid mimic, wherein the boramino acid mimic has the same structure as the amino acid except a COO⁻ moiety of the amino acid is replaced with a BF₃ moiety in the boramino acid mimic, wherein at least one fluorine is ¹⁸F,
   administering to a subject with a tumor an effective amount of the boramino acid mimic, and utilizing positron emission tomography to take an image of the tumor within the subject.
22. The method of aspect 21, wherein the subject is a mammal.
23. The method of aspect 22, wherein the mammal is a human.
24. The method of any one of aspects 21-23, wherein the method further comprises taking more than one image of the tumor, wherein the images are taken at different times, and measuring the size of the tumor on each image.
25. A process for the preparation of a compound or a pharmaceutically acceptable salt, solvate, or stereoisomer thereof, wherein the compound is of the Formula (I):

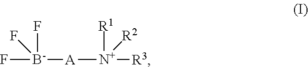

the process comprising reacting a compound of Formula (III)

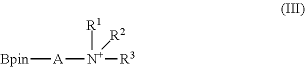

with fluorine under suitable conditions to produce a compound of Formula (I),
wherein
A is optionally substituted alkylenyl, wherein the substituents are selected from halo, alkoxyl, cycloalkyl, heterocycloalkyl, aryl, heteroaryl, hydroxyl, COOR², NR²R³, CONR²R³, SR², guanidino, alkynyl, and azido,
wherein each substituent cycloalkyl, heterocycloalkyl, aryl, and heteroaryl is optionally fused with one or more groups selected from cycloalkyl, heterocycloalkyl, aryl, and heteroaryl, wherein each substituent alkoxyl, cycloalkyl, heterocycloalkyl, aryl, and heteroaryl is optionally further substituted with one or more substituents selected from halo, alkyl, cycloalkyl, heterocycloalkyl, aryl, heteroaryl, hydroxyl, COOR², NR²R³, CONR²R³, SR², guanidino, alkynyl, and azido,
wherein substituent guanidino is optionally further substituted with one or more substituents selected from alkyl, cycloalkyl, heterocycloalkyl, aryl, and heteroaryl,
wherein each of R¹, R², and R³ is independently H or alkyl, the alkyl optionally substituted with one or more substituents selected from halo, alkoxyl, cycloalkyl, heterocycloalkyl, aryl, heteroaryl, hydroxyl, COOR², NR²R³, CONR²R³, SR², guanidino, alkynyl, and azido;
or
A and R¹ together with the nitrogen of the NR¹R²R³⁺ moiety form a heterocycloalkyl or heteroaryl ring, wherein the ring is optionally substituted with one or more substituents selected from halo, alkyl, alkoxyl, cycloalkyl, heterocycloalkyl, aryl, heteroaryl, hydroxyl, COOR², NR²R³, CONR²R³, SR², guanidino, alkynyl, and azido.
26. A process for the preparation of a compound, salt, solvate, or stereoisomer of aspect 6, the process comprising:
   (i) boronating a Grignard reagent comprising R⁴ under suitable conditions to produce a compound of R⁴—B(OMe)₂;
   (ii) reacting the R⁴—B(OMe)₂ with

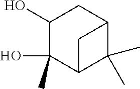

under suitable conditions to produce a compound of (P1):

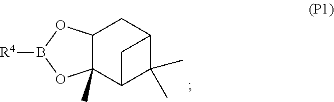

(iii) reacting (P1) with Li⁺CHCl₂⁻ under suitable conditions to produce a compound of (P2):

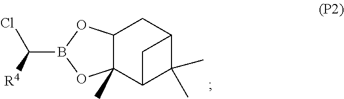

and
(iv) replacing the chlorine of (P2) with amino, the replacement optionally made by reacting (P2) with (Me₃Si)₂N⁻Li⁺ and HCl under suitable conditions to produce a compound of (P3):

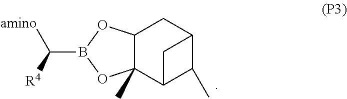

27. A process for the preparation of a compound, salt, solvate, or stereoisomer of aspect 6, the process comprising:
   (i) reacting R⁴CHO with H₂NSOC(CH₃)₃ under suitable conditions to produce a compound of R⁴CH═NSOC(CH₃)₃;
   (ii) boronating R⁴CH═NSOC(CH₃)₃ under suitable conditions to produce a compound of (P4):

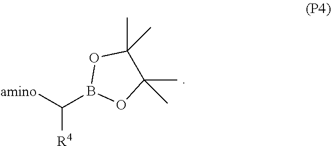

28. A process for the preparation of a compound, salt, solvate, or stereoisomer of aspect 6, the process comprising fluorinating the boron moiety of

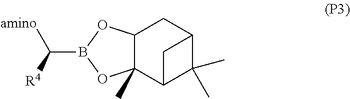

under suitable conditions to produce a compound of

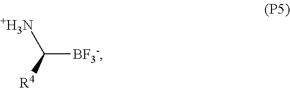

the fluorination optionally performed by reacting the (P3) with KF or KHF₂ and HCl.
29. A process for the preparation of a compound, salt, solvate, or stereoisomer of aspect 6, the process comprising fluorinating the boron moiety of

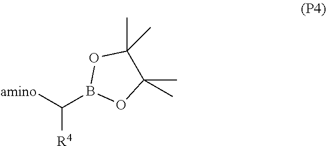

under suitable conditions to produce a compound of

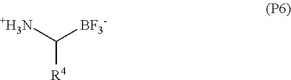

the fluorination optionally performed by reacting the (P4) with KF or KHF₂ and HCl.
30. A process for the preparation of a salt, solvate, or stereoisomer of aspect 14, the process comprising exchanging ¹⁹F for ¹⁸F under suitable conditions, the exchange optionally made by incubating the boramino acid with ¹⁸F-fluoride water.
31. A method of treating a tumor in a subject, the method comprising administering to the subject an effective amount of a compound, salt, solvate, or stereoisomer of any one of aspects 1-15 or a composition of aspect 16 and irradiating the subject with neutrons.

It shall be noted that the preceding are merely examples of embodiments. Other exemplary embodiments are apparent from the entirety of the description herein. It will also be understood by one of ordinary skill in the art that each of these embodiments may be used in various combinations with the other embodiments provided herein.

The following examples further illustrate the invention but, of course, should not be construed as in any way limiting its scope.

### Example 1

This example demonstrates similarities of free phenylalanine and Phe-BF₃ based on computer modeling studies, in accordance with embodiments of the invention.

Density functional theory (DFT) structure prediction of phenylalanine and phenylalanine mimics was performed, specifically investigating molecular electrostatic potential (MEP). The studied compounds were Phe, Phe-B(OH)2, Phe-BF₃, and Phe-B(OH)₃:

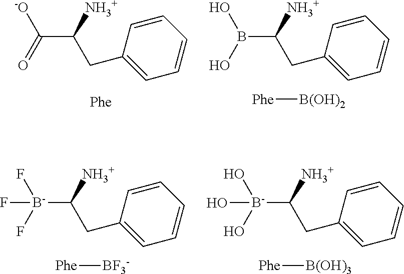

(Phe-B(OH)₂ and its hydrate Phe-B(OH)₃ are used in the clinic for boron neutron capture therapy (BNCT) to treat gliomas). The uptake of Phe-B(OH)₂ and Phe-B(OH)₃ in vivo involves the same channels as natural phenylalanine and is related to cancer cell replication. Phe-BF₃ shows mostly identical charge distribution compared with natural Phe and is more closely related to Phe than Phe-B(OH)₂ or Phe-B(OH)₃. This suggests that solution behavior of Phe-BF₃ would be very similar to Phe and that both compounds share in vivo properties. Further, since the carboxyl group is electronically similar to the trifluoroborate group and the amino groups are conserved between Phe and Phe-BF₃, it would be expected that both Phe and Phe-BF₃ would interact similarly with human LAT-1.

Under physiological conditions (pH=7.4), natural Leu is a ziwitterion since the alpha amine is protonated and positively charged, and the —COOH loses the acidic proton to be negatively charged. This unique electronic property is essential for its interaction with the LAT-1 transporter. For the BF₃⁻ moiety in ¹⁸F-Leu-BF₃, the average Mulliken charge on each fluorine is −0.416, so the total net charge on the surface of the BF₃⁻ moiety is −1.248. The average Mulliken charge on each oxygen is −0.640, meaning the total net charge on the —COO⁻ group is −1.280.

### Example 2

This example demonstrates the preparation of boramino acids, in accordance with embodiments of the invention.

Precursors were synthesized based on previously published methods. See Matteson et al., J. Am. Chem. Soc., 1986, 108:810-819 and Beenen et al., J. Am. Chem. Soc., 2008, 130:6910-1, both incorporated by reference herein in their entireties.

The precursors were directly fluorinated with KHF₂, and the chemical purity and identity were confirmed using high-resolution mass spectrometry (HRMS), HPLC chromatography, ¹⁹F/¹H-NMR, and X-ray crystallography.

For ¹⁸F-labeling, the one-step ¹⁸F-¹⁹F isotope exchange reaction (Liu et al., Angew. Chem. Int. Ed., 2014, 53:11876-80, incorporated by reference in its entirety) was used (product produced within 25 min) without the need for HPLC purification, giving 60±15% radiochemical yield (n>5 for each amino acid, non-decay corrected). The protocol is as follows: resuspend the boramino acid (2 μL stocking solution, 10 nmol) with pyridazine buffer (7.5 μL, 1 M, DMF:water=1:1, pH=2.5) in an Eppendorf tube (1.5 mL); add ¹⁸F-fluoride water (10 μL, 5-20 mCi) into the buffered solution; incubate the mixture under 60° C. for 10 min; quench the reaction by adding de-ionized water (2 mL), and load the crude directly on an activated Sep-Pak cartridge; remove the impurities by gently flushing the column with de-ionized water (2 mL), and elute the boramino acid with PBS/ethanol (0.5 mL, PBS:ethanol=1:1).

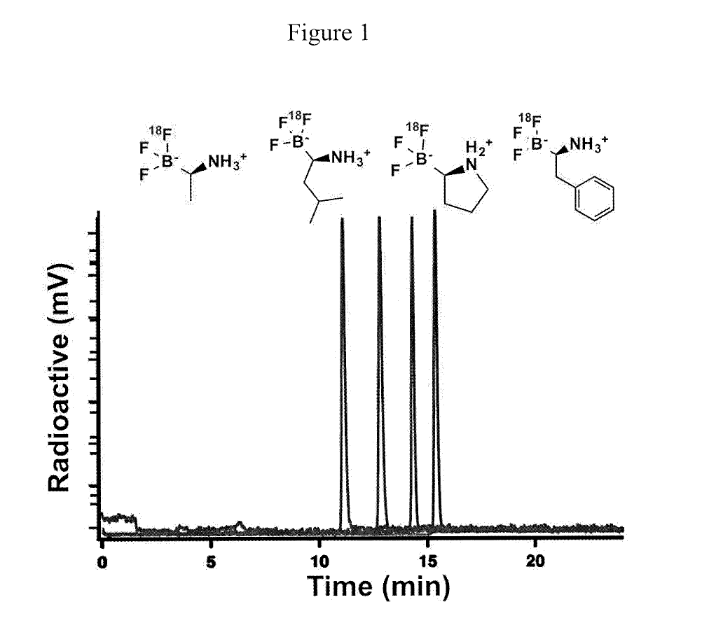

The chemical and radioactive purity of the resulting ¹⁸F-boramino acids were evaluated by injecting the reacted sample into an HPLC system. Only one major peak was found for each boramino acid in both radioactive and UV mode, demonstrating >98% radiochemical purity (FIG. 1). The specific activity was >37 GBq/μmol.

In a separate synthesis, starting with 10 nmol of ¹⁹F-Leu-BF₃ (˜2 μg) and 370-555 MBq (10-15 mCi) of ¹⁸F-fluoride, 222-370 MBq of ¹⁸F-Leu-BF₃ was obtained with the specific activity of 22.2-37.0 GBq/μmol (0.6-1.0 Ci/μmol), which is satisfactory for imaging the LAT-1 transporter. Starting with 2 μmol (˜300 μg) of ¹⁹F-Leu-BF₃ and 37 GBq (1 Ci) of ¹⁸F-fluoride, in 25 min ˜9.25 GBq (250 mCi) of ¹⁸F-Leu-BF₃ was achieved with high radiochemical purity (>98%). The specific activity was determined from dividing the amount of radiolabeled product by the amount of precursor ¹⁹F-Leu-BF₃, which is no less than 92.5 GBq/μmol (2.5 Ci/μmol, non decay corrected).

### Example 3

This example demonstrates specific uptake of boramino acids by amino acid transporters, in accordance with embodiments of the invention.

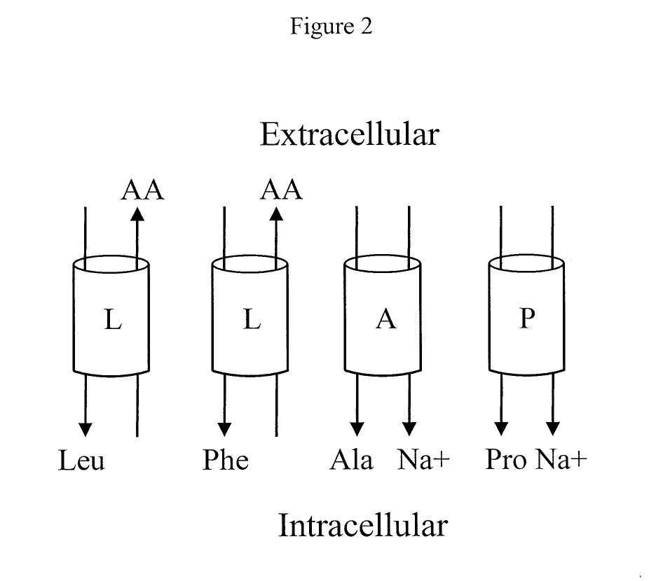

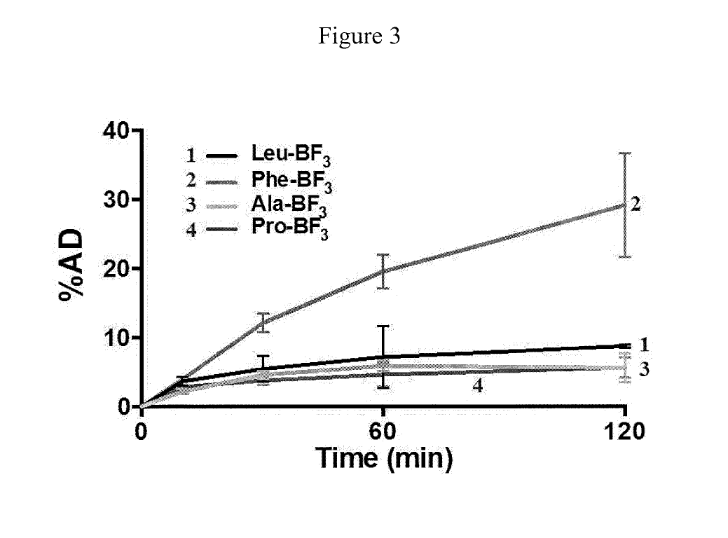

The cell uptake of ¹⁸F-boramino acids was evaluated by using U87MG human glioma cells, which are commonly used for studying tumor metabolism and amino acid uptake (see FIG. 2 and Table 2 for major amino acid transporters). The U87MG cells were in Dulbecco modified Eagle medium and Minimum Essential Medium under a humidified atmosphere with 5% CO₂ at 37° C. The culture media was supplemented with 10% fetal bovine serum, 100 U/mL penicillin, and 100 μg/mL streptomycin. Cells were cultured in 24-well plates at a density of 0.1 million cells per well and grown to 75% confluence. Prior to incubation, the cells were washed with phosphate-buffered saline (PBS). A final concentration of 2.0 μCi/mL ¹⁸F-BAA was added to wells (n=4) containing the cells, and the mixture was incubated under agitation at 37° C. for 15, 30, 60 and 120 min, respectively. The cells were then washed 3 times with ice-cold PBS, quenched with 0.1 M of NaOH and the cell-bound radioactivity was counted using a gamma counter. Uptake is defined as percentage of added radioactive dose (% AD). (See FIG. 3.)¹⁸F-boramino acids were successfully taken up by U87MG cells in a time-dependent manner, where the uptake for each BAA was distinct and essentially related to the identity of the side chain. For instance, the cell uptake of ¹⁸F-Phe-BF₃ was up to 28.5% AD at 120 min after incubation, whereas ¹⁸F-fluorodeoxyglucose (¹⁸F-FDG) had less than 5% AD cell uptake under the same condition. The cell uptake of ¹⁸F-Leu-BF₃ was time-dependent and increased to ˜7.5% AD at 60 min after incubation.

To demonstrate the specificity of boramino acid transportation, an in vitro competitive inhibition study was performed with U87MG cells in the presence of natural amino acids and transporter inhibitors at 25 mM, after incubation with BAA (about 2 nM) for 1 hour. The control is incubation with BAA only without competitor.

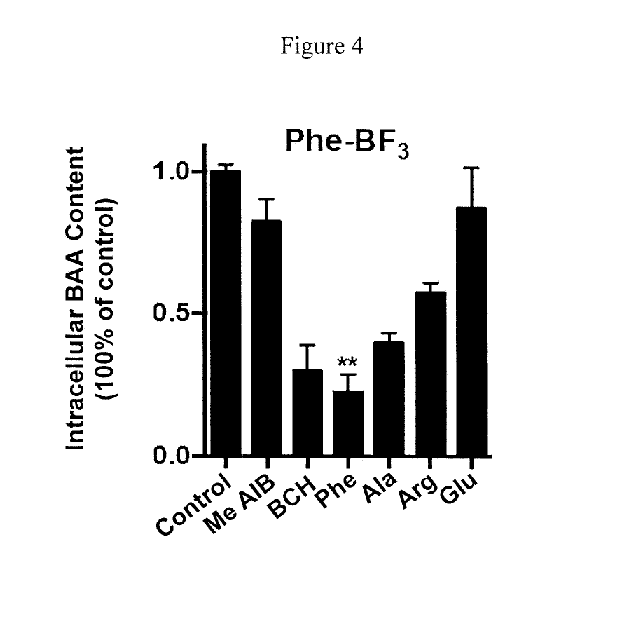

FIG. 4 shows the results for ¹⁸F-Phe-BF₃. After 60 mM incubation, L-phenylalanine showed nearly 80% inhibition of uptake of ¹⁸F-Phe-BF₃; and 2-amino-2-norbornanecarboxylic acid (BCH), which is a specific blocker for the L-type transporter, also showed significant inhibition (72.1%). L-Alanine showed moderate inhibition (60.3%, which is for both the A-type and ASC-type transporters). L-Arginine also demonstrated moderate inhibition as part of its uptake uses L-type transporters (42.6%). MeAIB (2-methylaminoisobutyric acid), which is a specific blocker for the A-type transporter, was used as the negative control and had less effect (17.6%). L-Glutamate, which is mainly taken up by cells specifically via X_{c}⁻-type transporters, exhibits less blocking as well (13.0%). These results suggest the cell uptake of ¹⁸F-Phe-BF₃ shares the same channel as phenylalanine and was essentially mediated by the system L amino acid transporter.

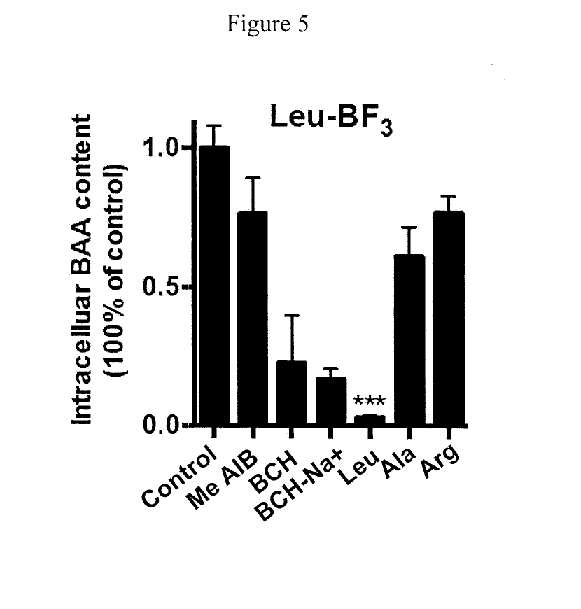

FIG. 5 shows the results for ¹⁸F-Leu-BF₃. Theoretically, the entry of ¹⁸F-Leu-BF₃ should take the same transporter as natural leucine, which demonstrates high preference for the L-type transporter but also can get through the A-type and ASC-type transporters. After 60 min incubation, L-leucine showed nearly 98% inhibition, and BCH also showed significant inhibition (77.4%). In addition, BCH with sodium-free medium, a typical inhibiting condition for leucine and its derivatives (Leu uptake known to be sodium-independent), demonstrated higher inhibition (83.0%) than only using BCH. L-Alanine showed moderate inhibition (38.9%) for the A-type and ASC-type transporter. L-Arginine demonstrated lower inhibition (23.0%) for leucine than for phenylalanine, possibly since the side chain of leucine is smaller in size. MeAIB, which is specific blocker for A-type transporter, served as a partial blocker (24.5%). These results demonstrate the cell uptake of ¹⁸F-Leu-BF₃ shares the same channel as leucine and was essentially mediated by the system L amino acid transporter.

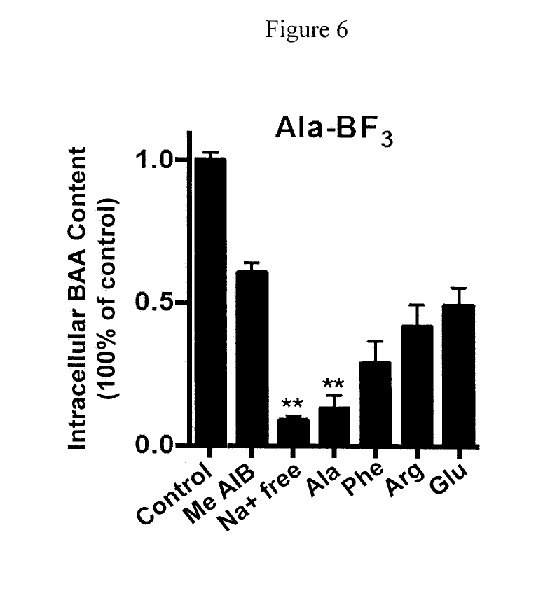

FIG. 6 shows the results for ¹⁸F-Ala-BF₃. Theoretically, the entry of ¹⁸F-Ala-BF₃ should take the same channel as natural occurring alanine, which demonstrates high preference for the A-type and ASC-type transporters, but also can get through the L-type transporter. After 60 min incubation, alanine showed nearly 90% inhibition, and sodium free medium, which is a specific blocking condition for A-type and ASC-type transporters, also showed significant inhibition (91.1%). MeAIB, which is a specific inhibitor for A-type transporter, served as a partial blocker (39.3%). L-phenylalanine showed effective inhibition (71.0%) for the uptake of ¹⁸F-Ala-BF₃ as it is the blocker for L-type and ASC-type transporter. L-Arginine showed moderate inhibition (58.2%) for the uptake of ¹⁸F-Ala-BF₃ as it can block the L-type and ASC-type transporter. L-Glutamate showed moderate inhibition (50.3%) for the uptake of ¹⁸F-Ala-BF₃ as it can be the blocker for L-type and other alanine-related transporters. These results suggest the cell uptake of ¹⁸F-Ala-BF₃ shares the same channel as alanine and was essentially mediated by system A and system ASC amino acid transporters.

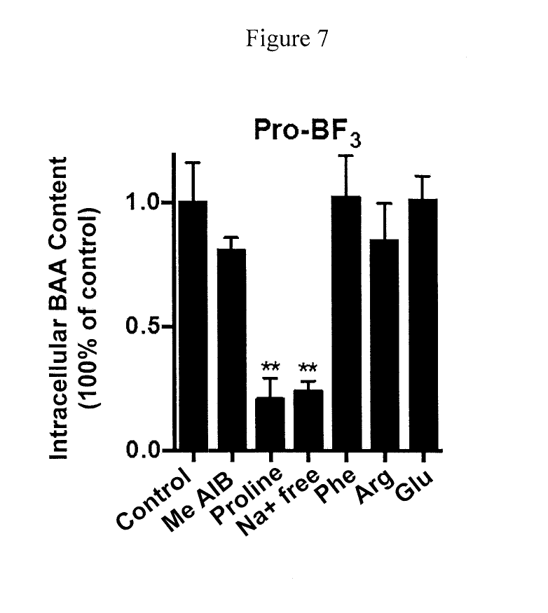

FIG. 7 shows the results for ¹⁸F-Pro-BF₃. Theoretically, the entry of ¹⁸F-Pro-BF₃ should take the same channel as natural occurring proline, which demonstrates high preference only for the P-type transporter (proline specific transporter). After 60 min incubation, proline showed nearly 80% inhibition, and sodium free medium, in which the P-type amino acid transporter should be inhibited, also showed significant inhibition (76.2%). MeAIB, which is a specific inhibitor for the A-type transporter, served as a partial blocker (39.3%). L-Phenylalanine, L-arginine, and L-glutamate, which are inhibitors for proline-irrelevant transporters, served as negative control and showed ineffective inhibition (0.1%, 15.6% and 0.2%, respectively). These results suggest the cell uptake of ¹⁸F-Pro-BF₃ shares the same channel as proline and is mediated by the system P amino acid transporter with high specificity.

### Example 4

This example further demonstrates specific uptake of a boramino acid, in accordance with embodiments of the invention.

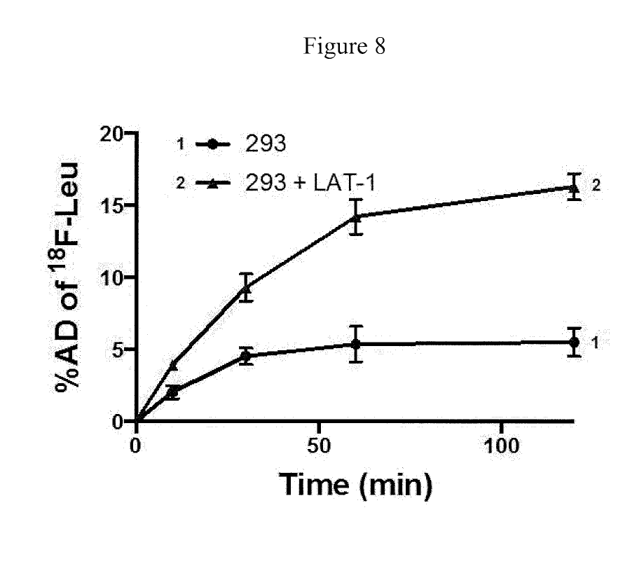

¹⁸F-Leu-BF₃ accumulated in HEK293 cells in a time-dependent manner, the cell uptake reached 10.4% AD at 120 min post incubation, and the uptake increased when incubated with LAT-1 up-regulated HEK293 cells (FIG. 8).

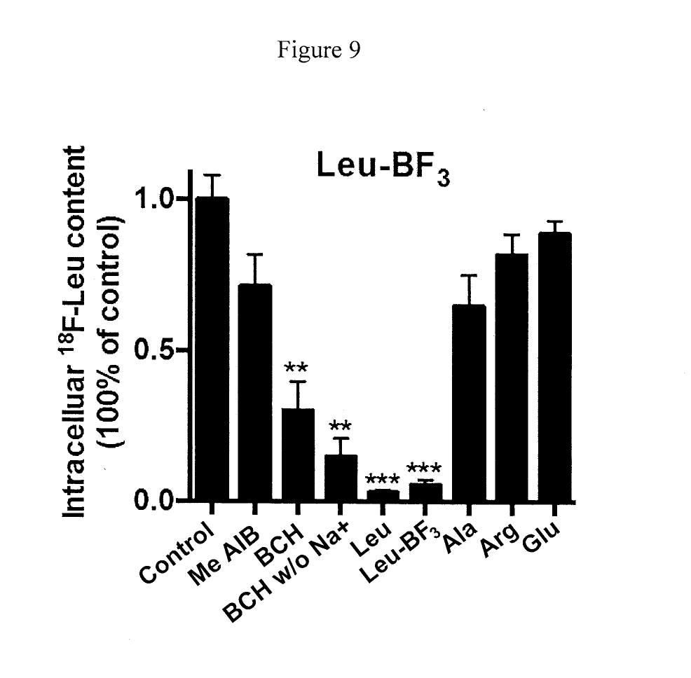

To study the specificity of BAA transportation, a competitive inhibition assay was performed using UM22B cells in the presence of natural amino acid and transporter inhibitor. After 60 min incubation, the cell uptake of ¹⁸F-Leu-BF₃ was substantially and selectively inhibited by natural leucine (94.7%) as well as BCH (83.0%) (FIG. 9).

The intracellular uptake of ¹⁸F-Leu-BF₃ is highly selective and competes with natural Leu. Cellular uptake of ¹⁸F-Leu-BF₃ increases when incubated with the cells that expresses more LAT-1. The entry of ¹⁸F-Leu-BF₃ is channel-selective and can be significantly blocked by natural Leu, BCH and ¹⁸F-Leu-BF₃ itself.

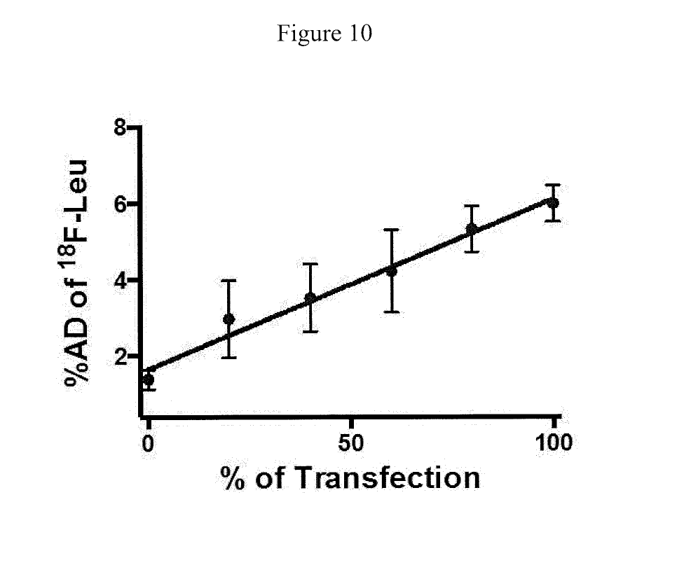

A nearly linear relationship is found between the cellular uptake of ¹⁸F-Leu-BF₃ in HEK293 cells and LAT-1 expression (FIG. 10).

### Example 5

This example illustrates the kinetics of uptake of boramino acids by amino acid transporters, in accordance with embodiments of the invention.

The uptake of BAA in U87MG cells was measured at increasing concentrations in phosphate-buffered saline (PBS) and plotted against the concentration of BAA. Prior to incubation, the cells were washed with phosphate-buffered saline (PBS) three times. The series of concentration-gradient solutions of Table 3 was added to wells (n=4), and the mixture was incubated under agitation at 37° C. for 60 min. The cells were then washed 3 times with ice-cold PBS, quenched with 0.1 M of NaOH and the cell-bound radioactivity was counted using a gamma counter. The K_{M} values of ¹⁸F-BAAs were calculated by Michaelis-Menten fitting using Igor Pro 6.22.

TABLE 3

| BAA | Concentrations (μM) |
|---|---|
| ¹⁸F-Phe-BF₃ | 3, 10, 30, 100, 300, 1000 |
| ¹⁸F-Leu-BF₃ | 0.3, 1, 3, 10, 30, 100, 300 |
| ¹⁸F-Ala-BF₃ | 1, 3, 10, 30, 100, 300, 1000 |
| ¹⁸F-Pro-BF₃ | 10, 30, 100, 400, 800, 1500, 2000, 3000, 6000, 10000 |

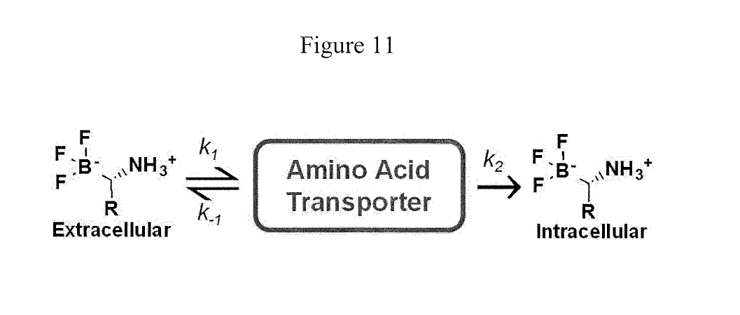

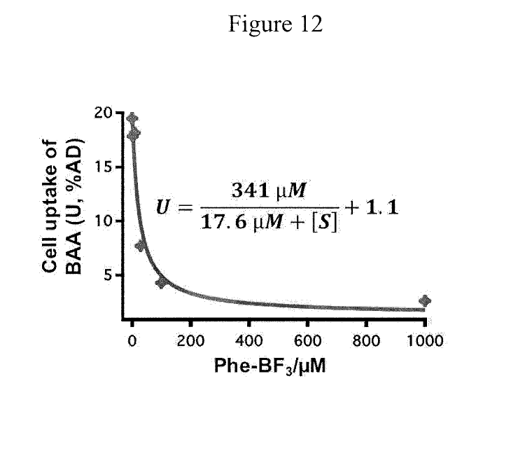

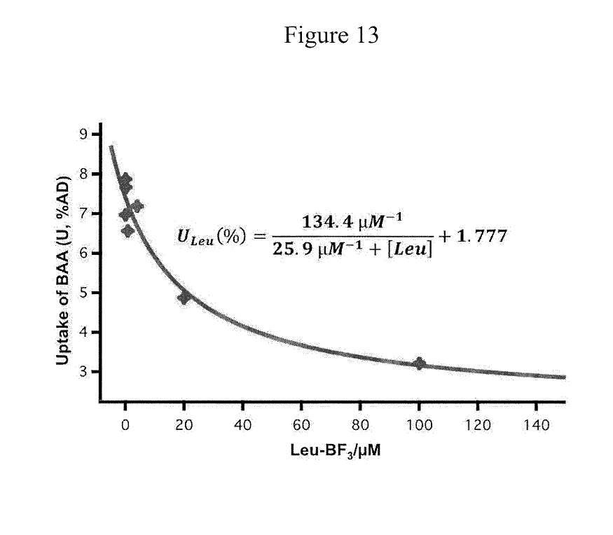

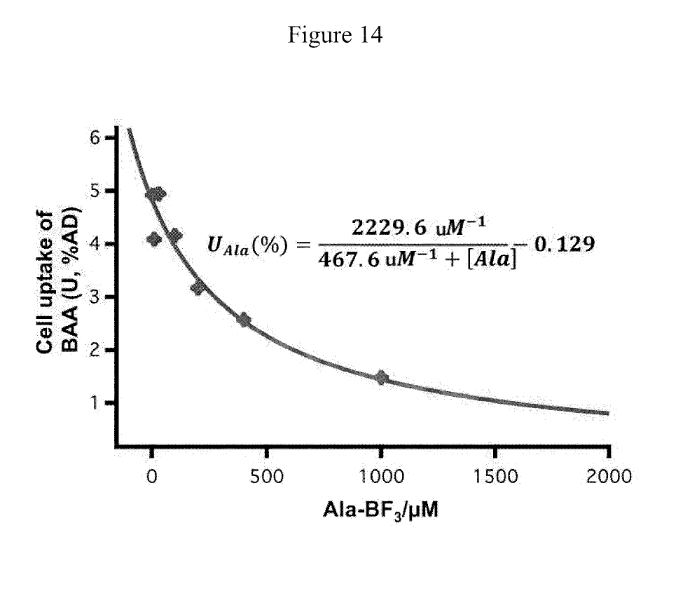

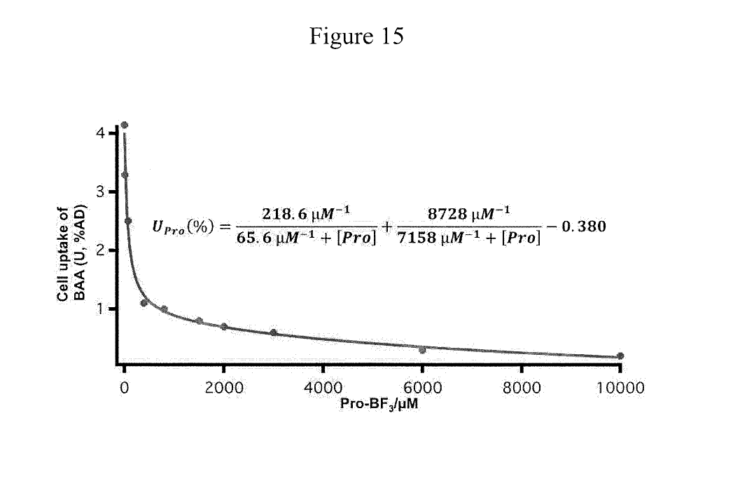

As illustrated in FIG. 11, the kinetics of amino acid transportation is an enzyme-mediated pathway that fit the Michaelis-Menten equation. The uptake-concentration correlations (FIGS. 12-15) fit well with Michaelis-Menten kinetics, giving the values of Table 4.

TABLE 4

K_{M}/μM

| | ¹⁸F-AA-BF₃ (BAA) | AA |
|---|---|---|
| Phe | 17.6 ± 4.2 | 18.7 ± 4.1 |
| Leu | 25.9 ± 2.4 | 19.7 ± 2.4 |
| Ala | 467.6 ± 87.2 | 370 |
| Pro | 65.6 ± 45.2 | 67 ± 11.5 |

The uptake of proline is biphasic since there are two main types of transporters for proline with about the same affinity. One can only transport proline, and has a higher binding affinity to proline (the P-type transporter). Proline also shares a different transporter with glycine (the G-type transporter), which has a weaker affinity for proline.

### Example 6

This example demonstrates the stability of a boramino acid, in accordance with embodiments of the invention.

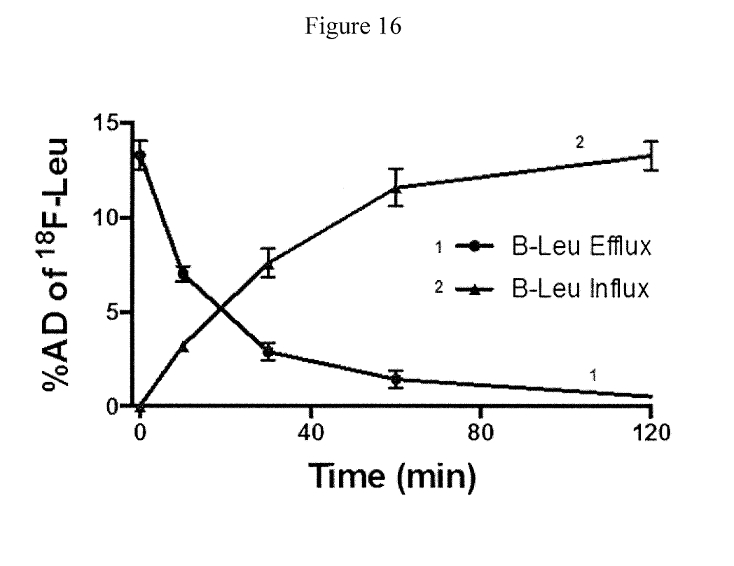

¹⁸F-Leu-BF₃ was first incubated with UM22B cells in PBS (phosphate buffered saline) for 120 min then was removed, followed by rapidly rinsing the cells with PBS three times. The cancer cells were incubated in medium essential medium for 10, 30, 60, and 120 min, and the residual radioactivity was measured (FIG. 16). The decreasing residual radioactivity is time-dependent, and most of the radioactive boramino acid was excreted from the cell in 90 min (less than 2% left at 120 min post medium incubation).

The radioactivity from efflux was further analyzed by re-injection into HPLC at 120 min (efflux: excreted from cell; influx: accumulated into cell). Only one peak was observed, and the elution time was corroborative to that for ¹⁸F-Leu-BF₃. The elution time here was 15 min, whereas in FIG. 1 the time was 12.5 min. After the data of FIG. 1 was obtained, maintenance was performed on the HPLC, after which the elution time of ¹⁸F-Leu-BF₃ on the HPLC was 15 min.

To further evaluate the metabolic stability of boramino acid, ¹⁸F-Leu-BF₃ was incubated with mouse plasma at 37° C. for 30 and 120 min. After incubation, most of the radioactivity was extracted from the plasma and followed by HPLC analysis. The defluoridation from boramino acid to boronic acid is almost negligible, as a single peak corroborative to that for ¹⁸F-Leu-BF₃ is observed.

To further evaluate its in vivo metabolic stability, ¹⁸F-Leu-BF₃ was also tested in living animals and followed by HPLC analysis of urine and blood samples. The observations suggested that ¹⁸F-Leu-BF₃ neither participates in protein synthesis nor loses fluoride during in vivo circulation.

Cell uptake of ¹⁸F-Leu-BF₃ specifically depends on the LAT-1 transporter. Since ¹⁸F-Leu-BF₃ is not involved in protein synthesis, in contrast to radiolabeled amino acids, and reflect transport only, this may give a simpler correlation between the tracer uptake to the expression level of amino acid transporter.

¹⁸F-Leu-BF₃ is not used by cells for protein synthesis and shows strong metabolic stability under both in vitro and in vivo conditions.

### Example 7

This example demonstrates that ¹⁸F-boramino acids exhibit high contrast PET imaging in tumor-bearing mice, in accordance with embodiments of the invention.

All imaging studies were performed as follows. PET scans were obtained and image analysis were performed using an Inveon small-animal PET scanner (Malvern, Pa., USA). About 3.7 MBq of ¹⁸F-AA were administered via tail vein injection under isoflurane anesthesia. For static acquisition, 10-min static PET scans were acquired at 30, 60 and 120 min after injection. For dynamic acquisition, 60 min dynamic PET scans were acquired, followed by a late-time-point scans at 2 h after tracer injection. With the acute inflammation model, 10-min static PET images were acquired at 1 h and 2 h after injection. The images were reconstructed using a 3-dimensional ordered subset expectation maximum algorithm, and no correction was applied for attenuation or scatter. For each scan, regions of interest (ROIs) were drawn using vendor software (ASI Pro 5.2.4.0; Siemens Medical Solutions) on decay-corrected whole-body coronal images. The radioactivity concentrations (accumulation) within the tumor, heart, muscle, liver, brain, and kidneys were obtained from mean pixel values within the multiple ROI volumes and then converted to megabecquerel per milliliter. These values were then divided by the administered activity to obtain (assuming a tissue density of 1 g/mL) an image-ROI-derived percentage injected dose per gram (% ID/g).

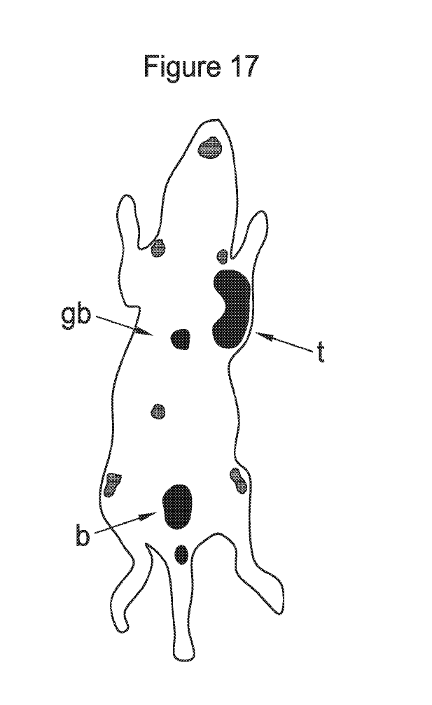

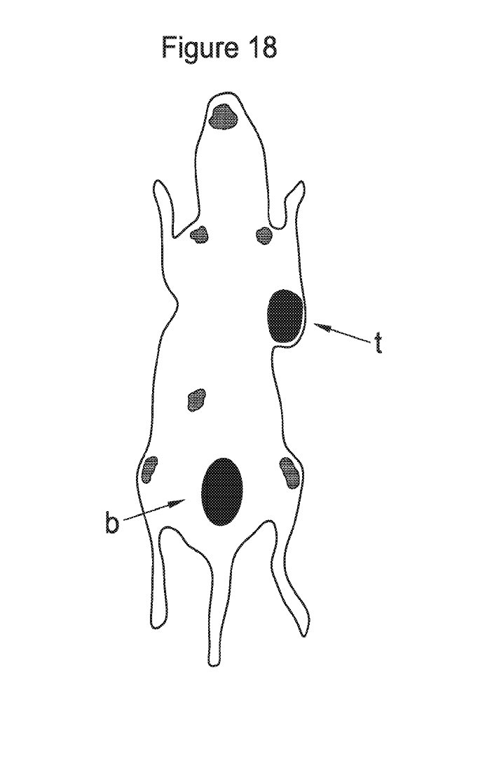

The ¹⁸F-Phe-BF₃ radiotracer was observed to specifically accumulate in the tumor of UM22B xenograft mice to give high tumor-to-background contrast at 120 min post injection (FIG. 17) as did ¹⁸F-Leu-BF₃ (FIG. 18). Both tracers specifically accumulated into tumors (t), whereas remainder cleared to bladders (b). Some gallbladder (gb) accumulation occurred for ¹⁸F-Phe-BF₃ because of its rapid hepatobiliary excretion. Kidney uptake was found to be almost negligible. ¹⁸F-Phe-BF₃ demonstrated equally high if not higher tumor-to-background ratio compared to ¹⁸F-FDG in tumor-bearing mice. Similar results were found with U87MG xenograft mice.

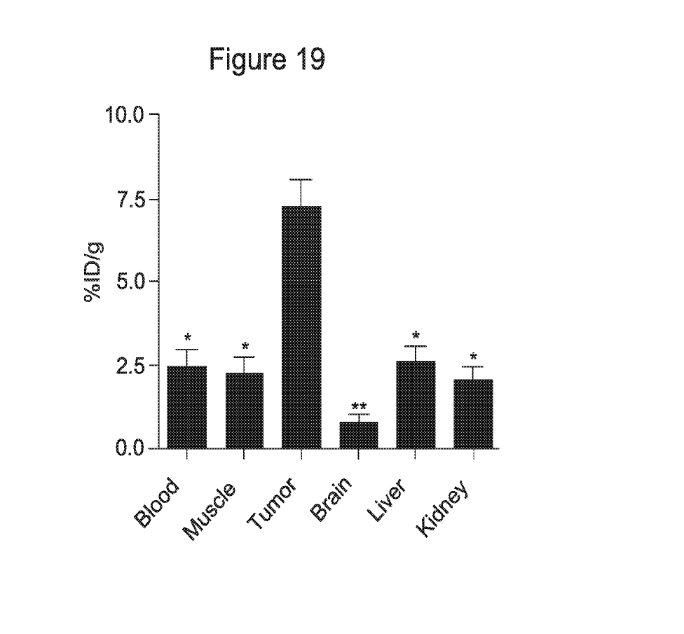

The biodistribution of ¹⁸F-Phe-BF₃ displayed higher uptake in tumors in comparison with normal tissues (FIG. 19). The average of the tumor uptake is given in Table 5 below.

TABLE 5

| | Average Uptake |
|---|---|
| Organ/Tissue | (% ID/g) |
| whole tumor region of interest | 7.31 ± 0.78 |
| hottest voxel cluster of tumor | 13.29 ± 1.18 |
| liver | 2.67 ± 0.44 |
| kidney | 2.13 ± 0.34 |
| blood pool | 2.46 ± 0.50 |
| muscle | 2.31 ± 0.44 |

For ¹⁸F-Leu-BF₃, the average uptake is given in Table 6.

TABLE 6

| | Average Uptake |
|---|---|
| Organ/Tissue | (% ID/g) |
| whole tumor region of interest | 6.72 ± 0.91 |
| hottest voxel cluster of tumor | 12.46 ± 3.0 |
| liver | 0.83 ± 0.16 |
| kidney | 2.32 ± 0.39 |
| blood pool | 0.47 ± 0.12 |
| muscle | 0.09 ± 0.03 |

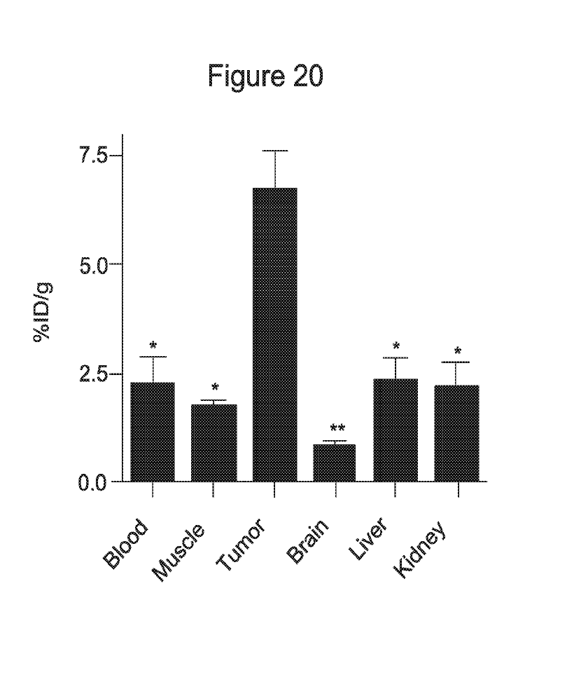

The biodistribution of ¹⁸F-Leu-BF₃ is shown in FIG. 20.

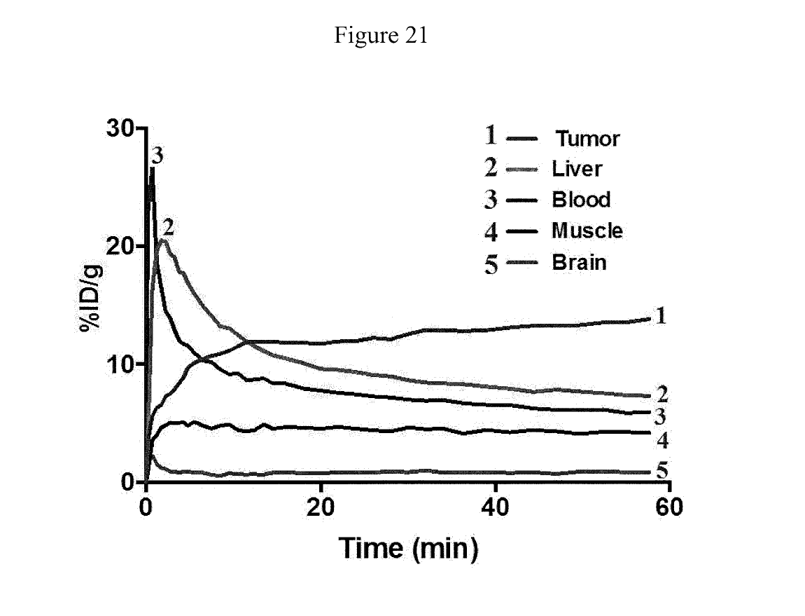

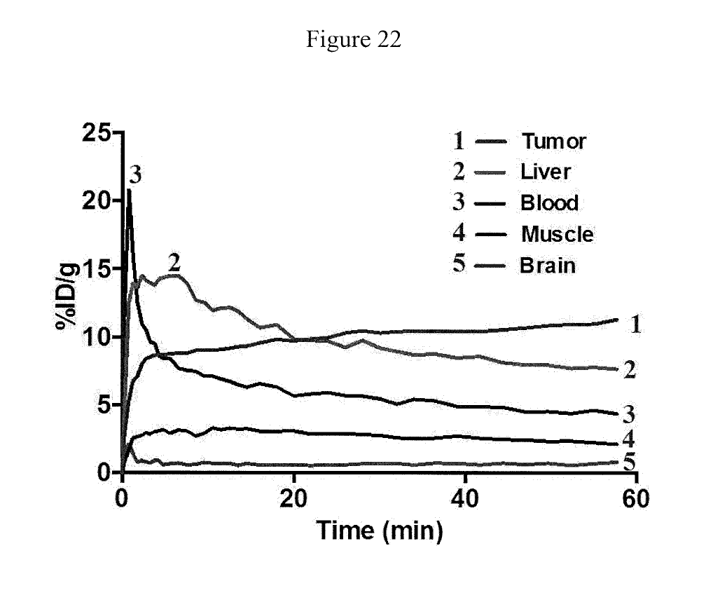

FIGS. 21 and 22 present time-activity curves of ¹⁸F-Phe-BF₃ uptake and ¹⁸F-Leu-BF₃ uptake, respectively, in tumor and other tissues from UM22B tumor-bearing mice. Time-dependent tumor uptake increased to a peak voxel cluster value of approximately 15% ID/g (percent injected dose per gram tissue) and 13% ID/g, respectively. Uptake in non-target tissues rapidly declined after reaching the peak value at a time point soon after intravenous administration.

For both ¹⁸F-Phe-BF₃ and ¹⁸F-Leu-BF₃, excretion was predominantly renal, with significant clearance to the bladder and low kidney retention. Some excretion via the hepatobiliary tract was noted and liver clearance was fast, leading to high tumor-to-liver ratios. Bone uptake was undetectable, and there was low background activity in blood and muscle, resulting in very high-contrast images.

The dynamic scan of ¹⁸F-Leu-BF₃ suggests that the radiotracer is re-concentrated to xenografts after whole body circulation, and the stability tests of blood and urine exhibit negligible metabolism of the radiotracer was observed during this re-concentration.

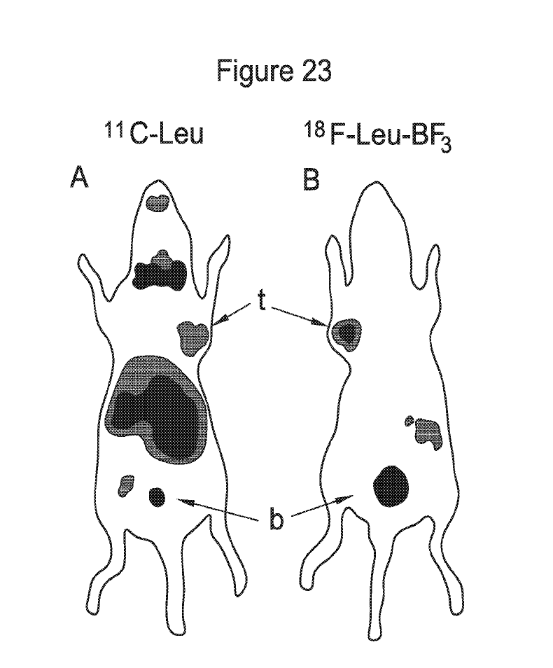

A comparison was made of ¹⁸F-Leu-BF₃ and ¹¹C-Leu. ¹⁸F-Leu-BF₃ exhibits intensive accumulation in tumor and notably lower uptake in major organs in comparison with natural Leu (FIG. 23). As shown, ¹⁸F-Leu-BF₃ demonstrates better tumor specificity than ¹¹C-Leu, whereas most of the remainder of ¹⁸F-Leu-BF₃ rapidly cleared to bladder through the renal system. This study was performed on the same animals (n=4) during the same day.

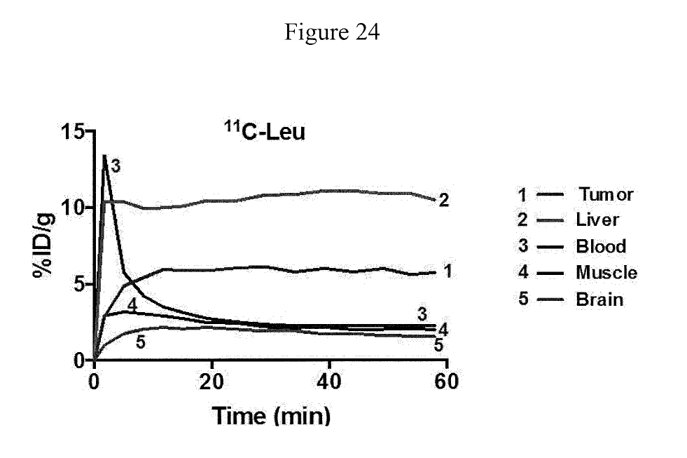

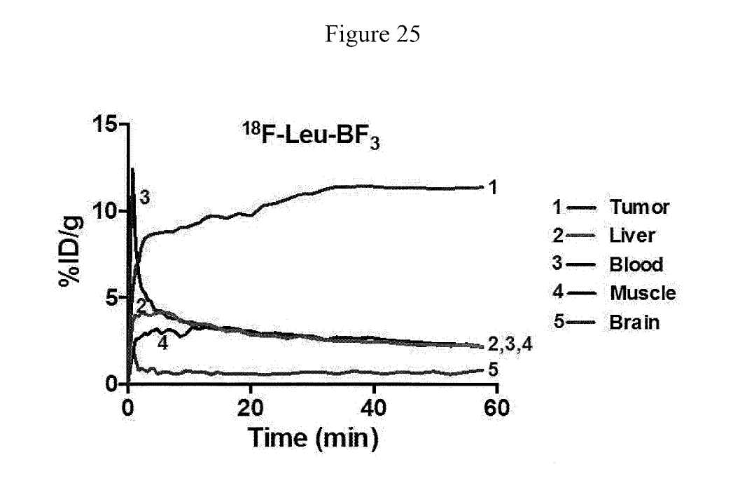

A time activity curve of the uptake of ¹¹C-Leu in tumor and other tissues from a tumor-bearing mouse is shown in FIG. 24. The time-dependent tumor uptake increased to a peak voxel cluster value of ˜6.5% ID/g in this particular mouse. A time activity curve of the uptake of ¹⁸F-Leu-BF₃ in tumor and other tissues from a tumor-bearing mouse is shown in FIG. 25. The time-dependent tumor uptake increased to a peak voxel cluster value of ˜6.5% ID/g in this particular mouse. Uptake in non-target tissues rapidly declined after reaching the peak value at an early time point soon after intravenous administration.

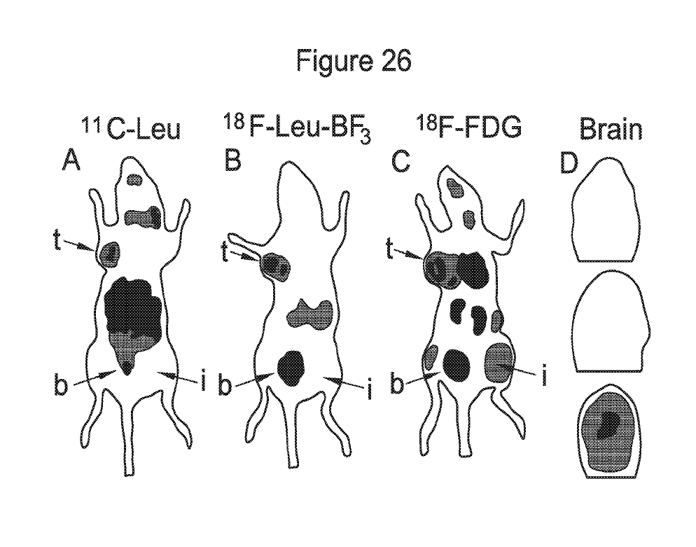

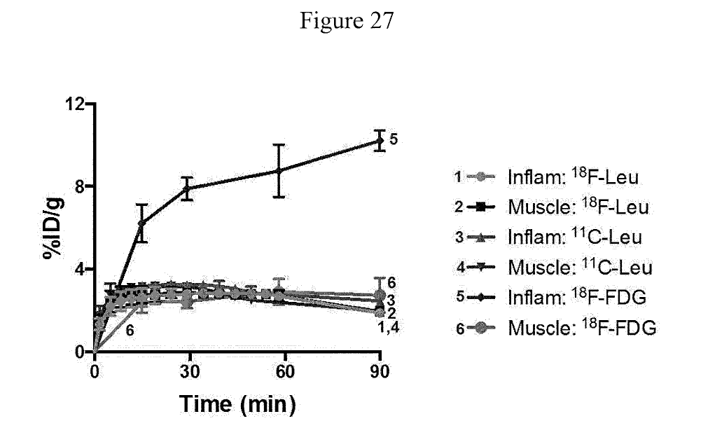

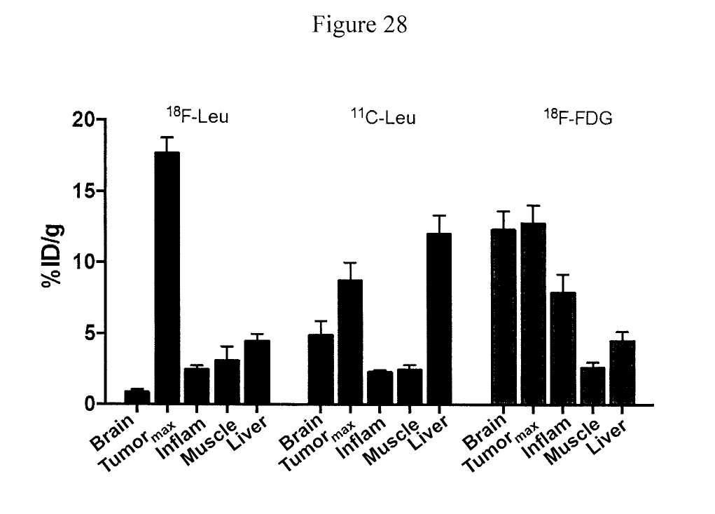

FIGS. 26-28 show that compared with ¹⁸F-FDG, ¹⁸F-Leu-BF₃ shows equal if not higher accumulation in tumor, but almost negligible uptake in inflammatory lesion.

### Example 8

This example demonstrates successful uptake of a boramino acid in an orthotopic xenografts transplant model.

The average uptake of orthotopic U87 human gliomas is 5.3±1.3% ID/g and the brain uptake is 0.5±0.2% ID/g at 60 min post injection, giving high contrast PET imaging for U87 tumor (tumor-to-brain ratio is above 10).

### Example 9

This example demonstrates boramino acids as theranostic boron delivery agents for imaging guided BNCT cancer treatment.

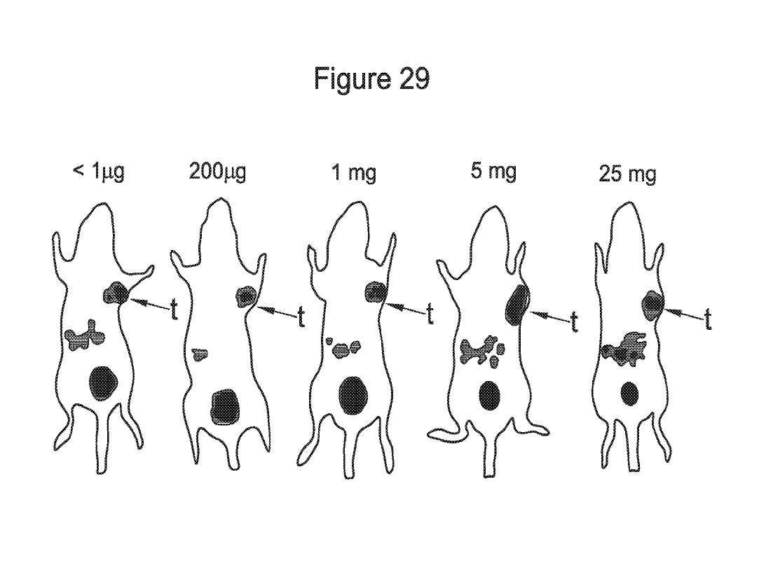

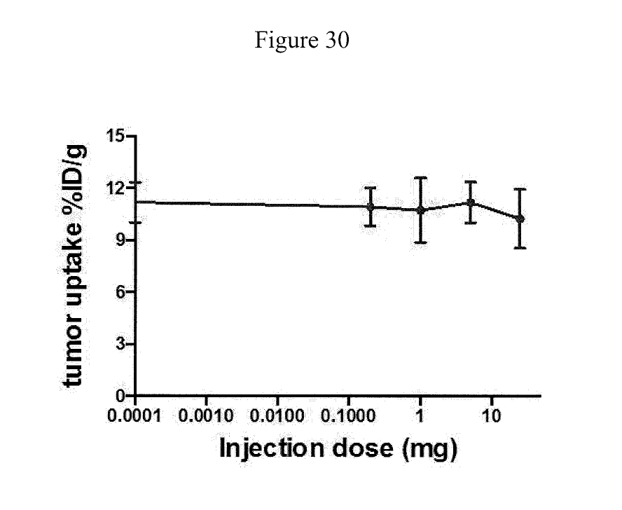

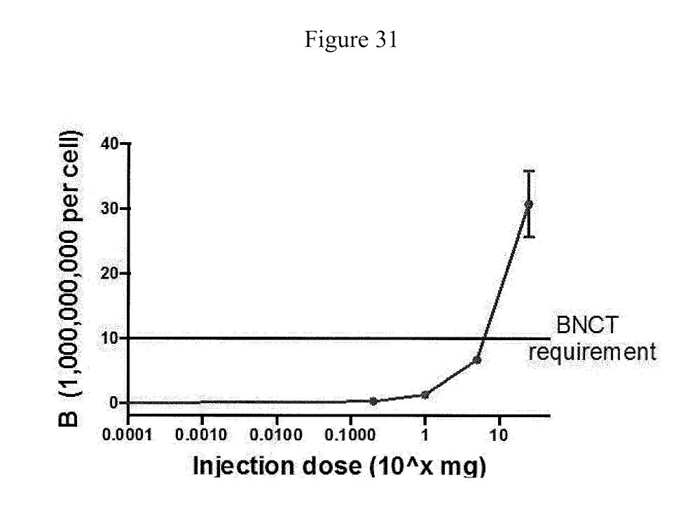

PET imaging was performed as in Example 7 in mice bearing UM22B xenografts. ¹⁸F-Leu-BF₃ was co-injected with <1 μg, 200 μg, 1 mg, 5 mg, or 25 mg of unlabeled Leu-BF₃. At 60 min post-injection, ¹⁸F-Leu-BF₃ showed high and consistent accumulation in UM22B tumor but demonstrates low uptake in the rest of the body (FIGS. 29-31). The tracer had predominant renal clearance but had low kidney retention.

Tumor-bearing mice were sacrificed at 60 min after injection of 10 mg of Leu-BF₃. UM22B xenografts are dissolve into nitric acid and the resulting solution are analyzed by ICP.

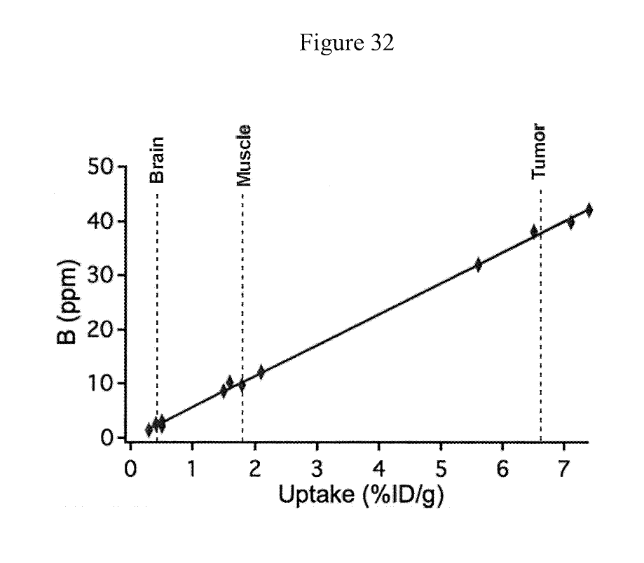

ICP analysis showed high boron accumulation in tumor with good selectivity in comparison with healthy tissues (FIG. 32). As a comparison, by injecting the same amount of 4-borono-L-phenylalanine (L-BPA), which is generally used for BNCT, only 11 ppm of boron in tumor (vs. 34.5 ppm of ¹⁸F-Leu-BF₃) and 1.8 of tumor-to-brain ratio (vs. 13.5 of ¹⁸F-Leu-BF₃) is observed from ICP analysis.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Also, everywhere “comprising” (or its equivalent) is recited, the “comprising” is considered to incorporate “consisting essentially of” and “consisting of.” Thus, an embodiment “comprising” (an) element(s) supports embodiments “consisting essentially of” and “consisting of” the recited element(s). Everywhere “consisting essentially of” is recited is considered to incorporate “consisting of.” Thus, an embodiment “consisting essentially of” (an) element(s) supports embodiments “consisting of” the recited element(s). Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of the Formula (I): 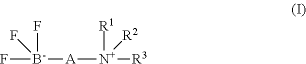
wherein
A is optionally substituted alkylenyl, wherein the substituents are selected from halo, alkoxyl, cycloalkyl, heterocycloalkyl, aryl, heteroaryl, hydroxyl, COOR², NR²R³, CONR²R³, SR², guanidino, alkynyl, and azido,
wherein each substituent cycloalkyl, heterocycloalkyl, aryl, and heteroaryl is optionally fused with one or more groups selected from cycloalkyl, heterocycloalkyl, aryl, and heteroaryl,
wherein each substituent alkoxyl, cycloalkyl, heterocycloalkyl, aryl, and heteroaryl is optionally further substituted with one or more substituents selected from halo, alkyl, cycloalkyl, heterocycloalkyl, aryl, heteroaryl, hydroxyl, COOR², NR²R³, CONR²R³, SR², guanidino, alkynyl, and azido,
wherein substituent guanidino is optionally further substituted with one or more substituents selected from alkyl, cycloalkyl, heterocycloalkyl, aryl, and heteroaryl,
wherein each of R¹, R², and R³ is independently H or alkyl, the alkyl optionally substituted with one or more substituents selected from halo, alkoxyl, cycloalkyl, heterocycloalkyl, aryl, heteroaryl, hydroxyl, COOR², NR²R³, CONR²R³, SR², guanidino, alkynyl, and azido;
or
A and R¹ together with the nitrogen of the NR¹R²R³⁺ moiety form a heterocycloalkyl or heteroaryl ring, wherein the ring is optionally substituted with one or more substituents selected from halo, alkyl, alkoxyl, cycloalkyl, heterocycloalkyl, aryl, heteroaryl, hydroxyl, COOR², NR²R³, CONR²R³, SR², guanidino, alkynyl, and azido;
or a pharmaceutically acceptable salt, solvate, or stereoisomer thereof.

2.-3. (canceled)

4. The compound, salt, solvate, or stereoisomer of claim 1, wherein A is methylenyl, ethylenyl, isobutylenyl, or isopentylenyl.

5. The compound, salt, solvate, or stereoisomer of claim 1, wherein the BF₃⁻ and NR¹R²R³⁺ moieties are attached to the same carbon of A.

6. The compound, salt, solvate, or stereoisomer of claim 1, wherein the compound is of the Formula (II): 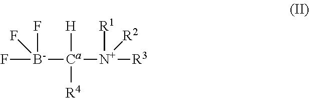
wherein
C^{a} is in the R or S configuration; and
R⁴ is H or alkyl, the alkyl optionally substituted with one or more substituents selected from halo, alkoxyl, cycloalkyl, heterocycloalkyl, aryl, heteroaryl, hydroxyl, COOR², NR²R³, CONR²R³, SR², guanidino, alkynyl, and azido,
wherein each substituent cycloalkyl, heterocycloalkyl, aryl, and heteroaryl is optionally fused with one or more groups selected from cycloalkyl, heterocycloalkyl, aryl, and heteroaryl,
wherein each substituent alkoxyl, cycloalkyl, heterocycloalkyl, aryl, and heteroaryl is optionally further substituted with one or more substituents selected from halo, alkyl, cycloalkyl, heterocycloalkyl, aryl, heteroaryl, hydroxyl, COOR², NR²R³, CONR²R³, SR², guanidino, alkynyl, and azido, and
wherein substituent guanidino is optionally further substituted with one or more substituents selected from alkyl, cycloalkyl, heterocycloalkyl, aryl, and heteroaryl; or
R⁴ and R¹ together with the nitrogen of the NR¹R²R³⁺ moiety form a heterocycloalkyl or heteroaryl ring, wherein the ring is optionally substituted with one or more substituents selected from halo, alkyl, alkoxyl, cycloalkyl, heterocycloalkyl, aryl, heteroaryl, hydroxyl, COOR², NR²R³, CONR²R³, SR², guanidino, alkynyl, and azido.

7. The compound, salt, solvate, or stereoisomer of claim 1, wherein the compound is one of the following: 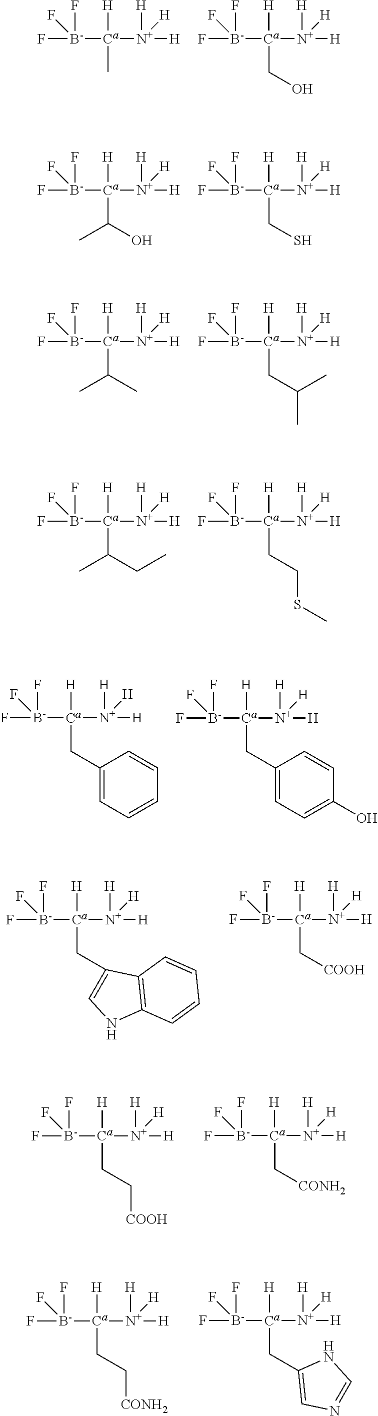 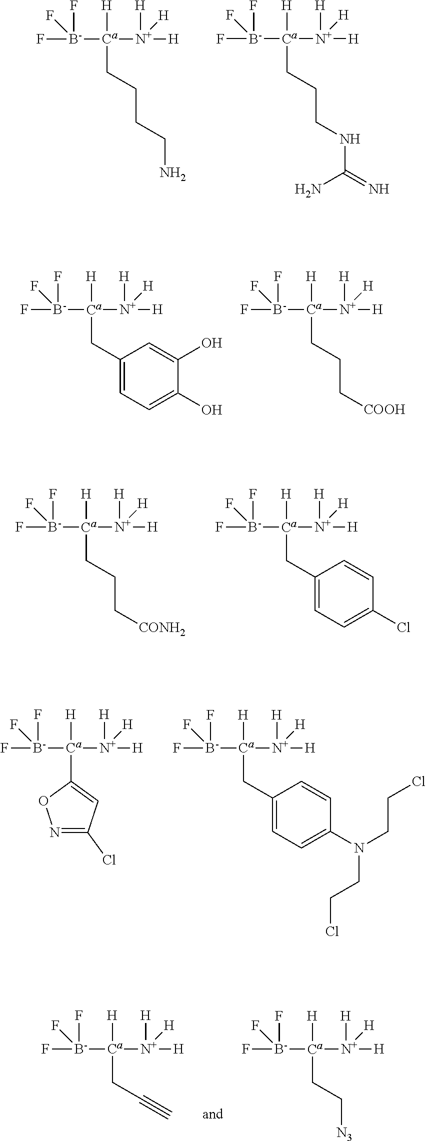
wherein C^{a} is in the R or S configuration.

8. The compound, salt, solvate, or stereoisomer of claim 1, wherein A and R¹ together with the nitrogen of the NR¹R²R³⁺ moiety form a heterocycloalkyl or heteroaryl ring.

9. The compound, salt, solvate, or stereoisomer of claim 6, wherein R⁴ and R¹ together with the nitrogen of the NR¹R²R³⁺ moiety form a heterocycloalkyl or heteroaryl ring.

10. The compound, salt, solvate, or stereoisomer of claim 8, wherein the compound is

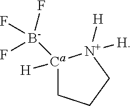

11.-12. (canceled)

13. The compound, salt, solvate, or stereoisomer of claim 1, wherein at least one F is ¹⁸F.

14. (canceled)

15. The compound, salt, solvate, or stereoisomer of claim 1, wherein the compound is one of the following:

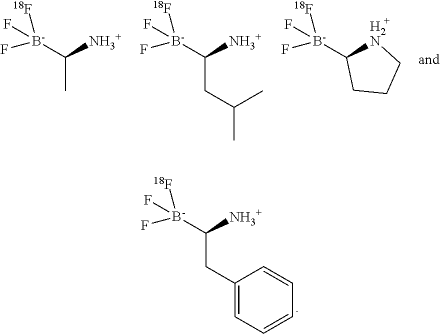

16. A composition comprising a compound, salt, solvate, or stereoisomer of claim 1 and a pharmaceutically acceptable carrier.

17. A method of imaging a tumor in a tissue, the method comprising utilizing positron emission tomography to take an image of a tissue having a tumor that has been exposed to a compound, salt, solvate, or stereoisomer of claim 1, wherein the compound, salt, solvate, or stereoisomer contains at least one ¹⁸F.

18.-19. (canceled)

20. The method of claim 17, wherein the method further comprises taking more than one image of the tissue, wherein the images are taken at different times, and measuring the size of the tumor in the tissue on each image.

21. A method of imaging tumor uptake in a tissue, the method comprising:
identifying an amino acid having at least one COO⁻ moiety,
generating a boramino acid mimic, wherein the boramino acid mimic has the same structure as the amino acid except a COO⁻ moiety of the amino acid is replaced with a BF₃⁻ moiety in the boramino acid mimic, wherein at least one fluorine is ¹⁸F,
exposing a tissue with a tumor to the boramino acid mimic, and
utilizing positron emission tomography to take an image of the tumor within the tissue.

22.-23. (canceled)

24. The method of claim 21, wherein the method further comprises taking more than one image of the tissue, wherein the images are taken at different times, and measuring the size of the tumor in the tissue on each image.

25. A process for the preparation of a compound or a pharmaceutically acceptable salt, solvate, or stereoisomer thereof, wherein the compound is of the Formula (I): 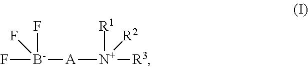
the process comprising reacting a compound of Formula (III) 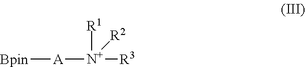
with fluorine to produce a compound of Formula (I),
wherein
Bpin is boronic acid pinacol ester;
A is optionally substituted alkylenyl, wherein the substituents are selected from halo, alkoxyl, cycloalkyl, heterocycloalkyl, aryl, heteroaryl, hydroxyl, COOR², NR²R³, CONR²R³, SR², guanidino, alkynyl, and azido,
wherein each substituent cycloalkyl, heterocycloalkyl, aryl, and heteroaryl is optionally fused with one or more groups selected from cycloalkyl, heterocycloalkyl, aryl, and heteroaryl,
wherein each substituent alkoxyl, cycloalkyl, heterocycloalkyl, aryl, and heteroaryl is optionally further substituted with one or more substituents selected from halo, alkyl, cycloalkyl, heterocycloalkyl, aryl, heteroaryl, hydroxyl, COOR², NR²R³, CONR²R³, SR², guanidino, alkynyl, and azido,
wherein substituent guanidino is optionally further substituted with one or more substituents selected from alkyl, cycloalkyl, heterocycloalkyl, aryl, and heteroaryl,
wherein each of R¹, R², and R³ is independently H or alkyl, the alkyl optionally substituted with one or more substituents selected from halo, alkoxyl, cycloalkyl, heterocycloalkyl, aryl, heteroaryl, hydroxyl, COOR², NR²R³, CONR²R³, SR², guanidino, alkynyl, and azido;
or
A and R¹ together with the nitrogen of the NR¹R²R³⁺ moiety form a heterocycloalkyl or heteroaryl ring, wherein the ring is optionally substituted with one or more substituents selected from halo, alkyl, alkoxyl, cycloalkyl, heterocycloalkyl, aryl, heteroaryl, hydroxyl, COOR², NR²R³, CONR²R³, SR², guanidino, alkynyl, and azido.

26. A process for the preparation of a compound, salt, solvate, or stereoisomer of claim 6, the process comprising:
(i) boronating a Grignard reagent comprising R⁴ to produce a compound of R⁴—B(OMe)₂;
(ii) reacting the R⁴—B(OMe)₂ with 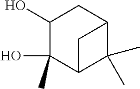
to produce a compound of formula: 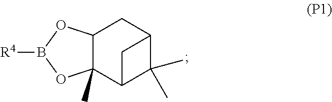
(iii) reacting (P1) with Li⁺CHCl₂⁻ to produce a compound of formula: 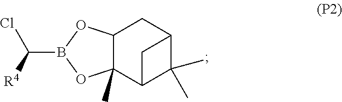
(iv) replacing the chlorine of (P2) with amino, the replacement optionally made by reacting (P2) with (Me₃Si)₂N⁻Li⁺ and HCl to produce a compound of formula: 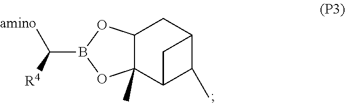
(v) fluorinating the boron moiety of (P3) to produce a compound of formula: 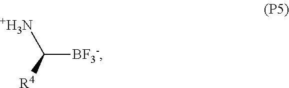
the fluorination optionally performed by reacting the compound of (P3) with KF or KHF₂ and HCl.

27. A process for the preparation of a compound, salt, solvate, or stereoisomer of claim 6, the process comprising:
(i) reacting R⁴CHO with H₂NSOC(CH₃)₃ to produce a compound of R⁴CH═NSOC(CH₃)₃;
(ii) boronating R⁴CH═NSOC(CH₃)₃ to produce a compound of formula: 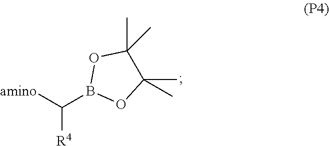
(iii) fluorinating the boron moiety of (P4) to produce a compound of formula: 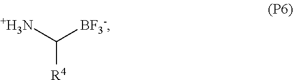
the fluorination optionally performed by reacting the compound of (P4) with KF or KHF₂ and HCl.

28. (canceled)

29. A method of treating a tumor in a subject, the method comprising administering to the subject an effective amount of a compound, salt, solvate, or stereoisomer of claim 1.

30. A method of boron neutron capture treatment of a tumor in a subject, the method comprising administering to the subject an effective amount of a compound, salt, solvate, or stereoisomer of claim 1.

31. A method of imaging a tumor within a subject, the method comprising administering to the subject an effect amount of a compound, salt, solvate, or stereoisomer of claim 1, wherein the compound, salt, solvate, or stereoisomer contains at least one ¹⁸F, and utilizing positron emission tomography to take an image of the tumor within the subject.

32. The method of claim 31, wherein the method further comprises taking more than one image of the tumor, wherein the images are taken at different times, and measuring the size of the tumor on each image.

33. A method of imaging tumor uptake, the method comprising
identifying an amino acid having at least one COO⁻ moiety,
generating a boramino acid mimic, wherein the boramino acid mimic has the same structure as the amino acid except a COO⁻ moiety of the amino acid is replaced with a BF₃⁻ moiety in the boramino acid mimic, wherein at least one fluorine is ¹⁸F,
administering to a subject with a tumor an effective amount of the boramino acid mimic, and
utilizing positron emission tomography to take an image of the tumor within the subject.

34. The method of claim 33, wherein the method further comprises taking more than one image of the tumor, wherein the images are taken at different times, and measuring the size of the tumor on each image.
